SEQUENCE LISTING (20 sequences)

SEQ ID NO: 1 - DNA, 1440 bp, Burkholderia sp.
atgacttaccgcaacgaatctgcctggatccagccggccgcccccaccaccacggccgccgcaccgcgcgcgacgcaggcgcgcaccaccgccgaataccgcgcgctcgacgccgcgcaccacatccatccgttctcggacatgggcgcgctgaaccgcgcgggcagccgcgtgatcgtgaaggccgacggcgtctacctgtgggactcggacggcaacaaggtgatcgacgggatggcgggcctctggtgcgtgaacgtcggctacggccgcaaggaactcgccgacgccgcgtatcgccagctccaggaactgccgttctacaacacgttcttcaagaccacgcacccgccggtgatcgagctttccgcgatgctcgcggaagtcacgccggccggcttcaaccacttcttctattgcaacagcggctcggaaggcaacgacaccgtgctgcgcctcgtgcaccagtactggcgcgtgcagggccagccgcagaagaagtatgtgatctcgcgcaagaacggttatcacggctcgacgatcgcgggcggcacgctcggcgggatgggctacatgcacgagcagatgccgtcgaaggtcgagaacatcgtgcacatcgaccagccgtatttcttcggcgaagcagcggccggcgaaacgccggaagcgttcggcctggcccgcgcgcagcagctcgaagcgaagatcctcgaactcggcgcggagaacgtcgccgcgttcatcggcgagccgttccagggcgccggcggcgtgatcttcccgccgtcgacgtactggccggaaatccagcggatctgtcgcaagtacgacatcctgctggtggccgacgaagtgatcggcggcttcggccgcaccggcgaatggttcgcgcaccagcacttcggcttcgagccggacctgatcacgatggccaagggcctcacgtcgggctacgtgccgatgggtgccgtcggcattcacgagcgcgtcgcgcggccgatcatcgacaacggcgaattcaaccacggcctcacgtactccggccacccggtggcggcagccgtcgcggtcgcgaacctgaagctgctgccgacgaagggatcgtcgagcgcgtgaagaacgacacgggcccgtacttcaggcgctgatgcgcgaaaccttcgcgcgtcacccgatcgtcggcgaagtgcacggccacggcctcgtcgcgagcctgcagctcgcggaagcgccggccgacgccgccgcttcgcgaacggcggcgacgtcggcacgatctgccgcgacttctgcttcaacggcaacctgatcatgcgtgcgaccggcgaccggatgctgctgtcgccgccgctcgtgatctcgcgtccggaaattgatgaactcgtatcgaaggcgaaaaaagcagtcgatgcaaccgcccagcaactgggtatttcgtaa

SEQ ID NO: 2 - protein, 480 aa, Burkholderia sp.
MTYRNESAWIQPAAPTTTAAAPRATQARTTAEYRALDAAHHIHPFSDMGALNRAGSRVIVKADGVYLWDSDGNKVIDGMAGLWCVNVGYGRKELADAAYRQLQELPFYNTFFKTTHPPVIELSAMLAEVTPAGFNHFFYCNSGSEGNDTVLRLVHQYWRVQGQPQKKYVISRKNGYHGSTIAGGTLGGMGYMHEQMPSKVENIVHIDQPYFFGEAAAGETPEAFGLARAQQLEAKILELGAENVAAFIGEPFQGAGGVIFPPSTYWPEIQRICRKYDILLVADEVIGGFGRTGEWFAHQHFGFEPDLITMAKGLTSGYVPMGAVGIHERVARPIIDNGEFNHGLTYSGHPVAAAVAVANLKLLRDEGIVERVKNDTGPYFQALMRETFARHPIVGEVHGHGLVASLQLAEAPAERRRFANGGDVGTICRDFCFNGNLIMRATGDRMLLSPPLVISRPEIDELVSKAKKAVDATAQQLGIS

SEQ ID NO: 3 - DNA, 2238 bp, Sporosarcina ureae
ttgaaaagtagagcagctgtagcatttaaacctggagaaccacttaaaattgtatacttttcatctcgtcgacatcgtaaatttggacctcttggtgaattttaacatgaaatcgatgtagaagaaccaaaagctaatgaggtattagtcaaaattctttacctttagctacatcttcttggttttcgattactccataatcagttttaagaaatgacttctgtctgtcatacagacgcatatacattatctggcgatgacgcagaaggatgaagacagacagtatgtctgcgtatatgtaatagaccgctactgcgtcttcctgtattccctgccgtgttaggtcatgaaggcgctggtgtcgtagtttcagtaggtcataagggacggcacaatccagtacttccgcgaccacagcatcaaagtcatccagaaggagtcacttccgttaaacctggcgatcacgtaattccactttacacacctcttcctcagtgaaggcaatttggaccgctagtgcattaaggtgaaatgtgtggagaatgcgggaaatgtaaattctgtctatccggtaaaactaacctatgcggtgcacttacgccctttacatttaagacagataggccattttgattggatacgccacgtattcgcgaaacacaaggtaaaggattaatgcctgacggtacgactcgcttctcttaagcgctttgtgttccatttcctaattacggactgccatgctgagcgaagagatacaatggagaacctatttatcactacatgggaacgagcacatttagtgagtatatgttacctcttggataaatagtgatgtacccttgctcgtgtaaatcactcataactgtcgtgtctgaaatctccttagttaaagtggaagaccacaacgtaccactttgacagcacagactttagaggaatcaatttcaccttctggtgttgcatggtgaagataaagtttgtctatttggatgtggcgtcactacgggtatcggggctgttcatctatttcaaacagataaacctacaccgcagtgatgcccatagccccgacaagtaaagactgcgaaagtagaagaaggtgcagttacagcagttttcggwctcggagctttctgacgctttcatcttcttccacgtcaatgtcgtcaaaagccwgagcctcgaattggtttggccgttatccaaggactaaagcaagcaaaagcaagccgtatcatttaaccaaaccggcaataggttcctgatttcgttcgttttcgttcggcatagtaagcaatcgatctcaatgaagataaatttgaactagcgaagaagatgggcgcaacacgttagctagagttacttctatttaaacttgatcgcttcttctacccgcgttgtgatttcatcaacccttctacattcgataagcccatccaagaagtcattgtagacctaaagtagttgggaagatgtaagctattcgggtaggttcttcagtaacatctgatgacggatggcggcgtagactacagtttcgagtgtatcggtaatgttgaagtctactgcctaccgccgcatctgatgtcaaagctcacatagccattacaacttcagatgaaagccgctcttgaatgctgccataaaggttggggcgagagtgtcatcatttactttcggcgagaacttacgacggtatttccaaccccgctctcacagtagtaaggcgttgcaggtgctggtaaagaaattcatactcgtcccttccaactagttacaccgcaacgtccacgaccatttctttaagtatgagcagggaaggttgatcaatgtggtcgcgtatggcgcggttctgcatttggtggagtaaaaggcagaaccgaattgccagcgcataccgcgccaagacgtaaaccacctcattttccgtcttggcttaacccagggatgattgatgactacatggacggtgaaattgatttagattcattcattggtccctactaactactgatgtacctgccactttaactaaatctaagtaagtaaacacaccaactaaacttcacggatatcaatgaagcatttgatttactgcataaatgtgtggttgatttgaagtgcctatagttacttcgtaaactaaatgacgtatttggtgaatcaattcgatcaatactaacatatggagagtgaccacttagttaagctagttatgattgtatacctctcact

SEQ ID NO: 4 - protein, 372 aa, Sporosarcina ureae
MKSRAAVAFKPGEPLKIVEIDVEEPKANEVLVKILYTSVCHTDAYTLSGDDAEGVFPAVLGHEGAGVVVSVGEGVTSVKPGDHVIPLYTPECGKCKFCLSGKTNLCGAIRETQGKGLMPDGTTRFSYNGEPIYHYMGTSTFSEYTVVSEISLVKVEDHNVPLDKVCLFGCGVTTGIGAVHKTAKVEEGAVTAVFGLGAIGLAVIQGLKQAKASRIIAIDLNEDKFELAKKMGATDFINPSTFDKPIQEVIVDMTDGGVDYSFECIGNVEVMKAALECCHKGWGESVIIGVAGAGKEIHTRPFQLVTGRVWRGSAFGGVKGRTELPGMIDDYMDGEIDLDSFITHQLNFTDINEAFDLLHKGESIRSILTYGE

SEQ ID NO: 5 - DNA, 20 bp, Burkholderia sp.
  misc_feature (3)..(3): n is i
  misc_feature (6)..(6): n is i
  misc_feature (12)..(12): n is i
  misc_feature (15)..(15): n is i
gcnccncaygcnacncargc

SEQ ID NO: 6 - DNA, 20 bp, Burkholderia sp.
  misc_feature (3)..(3): n is i
  misc_feature (6)..(6): n is i
  misc_feature (12)..(12): n is i
  misc_feature (15)..(15): n is i
tangcngcrtcngcngrytc

SEQ ID NO: 7 - DNA, 26 bp, Burkholderia sp.
  misc_feature (6)..(6): n is i
  misc_feature (12)..(12): n is i
  misc_feature (15)..(15): n is i
  misc_feature (21)..(21): n is i
aargcngayggngtntayycntggga

SEQ ID NO: 8 - DNA, 23 bp, Burkholderia sp.
  misc_feature (9)..(9): n is i
  misc_feature (12)..(12): n is i
  misc_feature (18)..(18): n is i
  misc_feature (21)..(21): n is i
aargarycngcngaygcngcnta

SEQ ID NO: 9 - DNA, 20 bp, Burkholderia sp.
  misc_feature (6)..(6): n is i
  misc_feature (12)..(12): n is i
crytcnacdatnccytcrtc

SEQ ID NO: 10 - DNA, 25 bp, Burkholderia cepacia
gggcttcaaccacttcttctattgc

SEQ ID NO: 11 - DNA, 23 bp, Burkholderia cepacia
gccgaagtgctggtgtgcgaacc

SEQ ID NO: 12 - DNA, 40 bp, Burkholderia sp.
gacatatttaaatcatatgacttaccgcaacgaatctgcc

SEQ ID NO: 13 - DNA, 41 bp, Burkholderia sp.
gatttaaatcccgggttacgaaatacccagttgctgggcgg

SEQ ID NO: 14 - protein, 18 aa, Sporosarcina ureae
  misc_feature (2)..(2): Xaa can be any naturally occurring amino acid
  misc_feature (4)..(4): Xaa can be any naturally occurring amino acid
  misc_feature (16)..(16): Xaa can be any naturally occurring amino acid
MXSXAAVAFKPGEPLXIV

SEQ ID NO: 15 - DNA, 23 bp, Sporosarcina ureae
  misc_feature (12)..(12): n is i
  misc_feature (21)..(21): n is i
garathgaygtngargarccnaa

SEQ ID NO: 16 - DNA, 20 bp, Sporosarcina ureae
  misc_feature (3)..(3): n is i
  misc_feature (6)..(6): n is i
  misc_feature (9)..(9): n is i
  misc_feature (18)..(18): n is i
ccngtnacnarytgraangg

SEQ ID NO: 17 - DNA, 23 bp, Sporosarcina ureae
caccagcgccttcatgacctaac

SEQ ID NO: 18 - DNA, 19 bp, Sporosarcina ureae
gccgctcttgaatgctgcc

SEQ ID NO: 19 - DNA, 39 bp, Sporosarcina ureae
gacatatttaaatcatatgaaaagtagagcagctgtagc

SEQ ID NO: 20 - DNA, 45 bp, Sporosarcina ureae
catgatttaaatggatcctcactctccataagttagtattgatcg

(19) United States
(12) Patent Application Publication
(10) Pub. No.: US 2007/0231858 A1
(43) Pub. Date: Oct. 4, 2007

(54) ENZYMATIC PREPARATION OF (S) AMINO ACID FROM (R,S) AMINO ACID OR FROM KETO ACID

(75) Inventors: Ramesh N. Patel, Bridgewater, NJ (US); Yijun Chen, Belle Mead, NJ (US); Steven L. Goldberg, Basking Ridge, NJ (US); Iqbal Gill, Denville, NJ (US); Animesh Goswami, Plainsboro, NJ (US); Thomas P. Tully, Middlesex, NJ (US); William L. Parker, Pennington, NJ (US); Ronald L. Hanson, Morris Plains, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/690,159

(22) Filed: Mar. 23, 2007

Related U.S. Application Data
(60) Provisional application No. 60/784,996, filed on Mar. 23, 2006.

(51) Int. Cl. C12P 21/06; C12P 13/04; C12N 9/90; C07D 213/55

(52) U.S. Cl. 435/69.1; 435/320.1; 435/325; 435/106; 435/233; 546/335

(57) ABSTRACT

Disclosed and claimed herein are novel biocatalysts for converting racemic mixtures of amino acids to an enantiomerically pure (S) form of the amino acid, methods for their use, and the enantiomerically enriched products of such biocatalytic processes.

This application claims priority to U.S. Application Ser. No. 60/784,996, filed Mar. 23, 2006.

### BACKGROUND OF THE INVENTION

The subject matter disclosed and claimed herein relates to enantiomerically purified (S) amino acids, methods for preparing such purified (S) amino acids, intermediates in such methods, and reagents for carrying out such methods.

Chirality is a factor to be considered with respect to the efficacy of many drugs and agrochemicals. The production of single enantiomers of chiral intermediates has become increasingly important. Single enantiomers can be produced by chemical or chemoenzymatic synthesis, and biocatalysis, the latter being the emphasis herein. Biocatalysis has many advantages over chemical synthesis which include the enantioselective and regioselective nature of enzyme-catalyzed reactions, and the ability of biocatalysts to carry out biocatalytic reactions at ambient temperature and atmospheric pressure. Biocatalysts avoid problems in isomerization, racemization, epimerization, and rearrangement often associated with the use of extreme conditions in chemical syntheses. Furthermore, microbial cells expressing an enzyme of interest, and the enzymes themselves, can be immobilized and reused for multiple biocatalytic reactions. The enzymes may be over-expressed to make biocatalytic processes economically efficient, and enzymes with modified activity/properties can be readily made by recombinant techniques.

Thus, there is a need in the art for such biocatalysts, methods for their use, and the enantiomerically enriched products of such biocatalyst use.

### SUMMARY OF THE INVENTION

One embodiment of the subject matter disclosed and claimed herein is an isolated 2-S-amino-3-[3-{6-(2-methylphenyl)}pyridyl]-propionic acid [BMS-728884], or a salt thereof.

A second embodiment is the use of isolated S-amino-3-[3-{6-(2-methylphenyl)}pyridyl]-propionic acid [BMS-728884] or a salt thereof, for the preparation of pharmaceuticals, such as glucagon-like peptide-1 (“GLP-1”) mimics or GLP-1 receptor modulators.

Another embodiment is 2-oxo-3[3-{6-(2-methylphenyl)}pyridyl]-propionic acid, or a salt thereof.

An additional embodiment is a substantially purified polypeptide, comprising, or consisting of, an amino acid sequence of SEQ ID NO:2 or SEQ ID NO:4, or functional equivalents thereof.

A further embodiment is an isolated nucleic acid comprising, or consisting of, a nucleic acid that encodes a polypeptide of SEQ ID NO:2 or SEQ ID NO:4, or functional equivalents thereof. In a preferred embodiment, the nucleic acid comprises the sequence of SEQ ID NO:1 or SEQ ID NO:3.

Another embodiment is a recombinant expression vector comprising isolated nucleic acids of the invention.

Additional embodiments include: recombinant host cells transfected with a recombinant expression vector comprising a nucleic acid that encodes a polypeptide of SEQ ID NO:2 and/or SEQ ID NO:4; and a cell extract derived from such recombinant host cells.

Other embodiments include a method for converting a racemic amino acid to an (S) amino acid, comprising (a) reacting a racemic amino acid with an amount of an amino acid oxidase (e.g., R-amino oxidase) to produce a mixture of (S) amino acid and keto acid; and (b) reacting the mixture with (i) an amount of an aminotransferase of SEQ ID NO:2 in the presence of an amino acid, or (ii) an amino acid dehydrogenase of SEQ ID NO:4 in the presence of an amino donor group such as ammonia or ammonium ion, and a cofactor selected from the group consisting of NADH; NAD+formate+formate dehydrogenase; and NAD+glucose+glucose dehydrogenase, to produce the (S) amino acid.

Additional methods include methods for converting a keto acid to an (S) amino acid, comprising reacting a keto acid of Formula I: 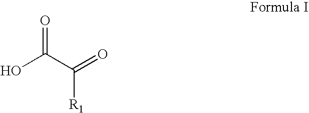
with (i) an amount of an aminotransferase of SEQ ID NO:2, in the presence of an amino acid, or (ii) an amino acid dehydrogenase of SEQ ID NO:4 in the presence of an amine donor (e.g. ammonia or an ammonium ion) to produce an (S) amino acid, wherein R₁ is selected from the group consisting of alkenyl, alkyl, alkynyl, aryl, arylalkyl, cycloalkyl, cycloalkylalkyl, heteroaryl, and heteroarylalkyl.

In a preferred embodiment, R₁ is heteroarylalkyl wherein the heteroaryl is pyridinyl optionally substituted with 2-methylphenyl.

A further embodiment is a method for converting 2-RS-amino-3-[3-{6-(2-methylphenyl)}pyridyl]-propionic acid to S-amino-[3-{6-(2-methylphenyl)}pyridyl]-propionic acid, comprising reacting 2-RS-amino-3-[3-{6-(2-methylphenyl)}pyridyl]-propionic acid with an amino acid oxidase in combination with a non-enzymatic reducing agent, or a salt thereof, in the presence of an inorganic catalyst, to produce S-amino-[3-{6-(2-methylphenyl)}pyridyl]-propionic and an imine form of amino acid, which is converted back to a racemic amino acid. In the overall process, a racemic amino acid is converted completely to an (S)-amino acid.

### BRIEF DESCRIPTION OF THE FIGURES

FIG. 1 provides a synthetic scheme for converting 2-RS-amino-3-[3-{6-(2-methylphenyl)}pyridyl]-propionic acid (BMS-700756) to S-amino-3-[3-{6-(2-methylphenyl)}pyridyl]-propionic acid (BMS-728884), using D-amino acid oxidase and a transaminase.

FIG. 2 provides a flow chart for synthesis of BMS-728884.

FIGS. 3A and 3B provide the DNA and amino acid sequence for adenosylylmethionine-8-amino-7-oxononanoate aminotransferase (AAOA) from Burkholderia species (SEQ ID NOS: 1 and 2).

FIG. 4 provides a synthetic scheme for converting racemic BMS-700756 into BMS-728884 using D-amino acid oxidase and an amino acid dehydrogenase.

FIGS. 5A and 5B provide the DNA and amino acid sequence for a amino acid dehydrogenase from Sporosarcina ureae (SEQ ID NOS: 3-4).

FIG. 6 is a synthetic scheme for converting racemic BMS-700756 into BMS-728884.

### DETAILED DESCRIPTION OF THE INVENTION

As used in the specification and the appended claims, the singular forms “a,” “an” and “the” include plural referents unless the context clearly dictates otherwise.

Unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

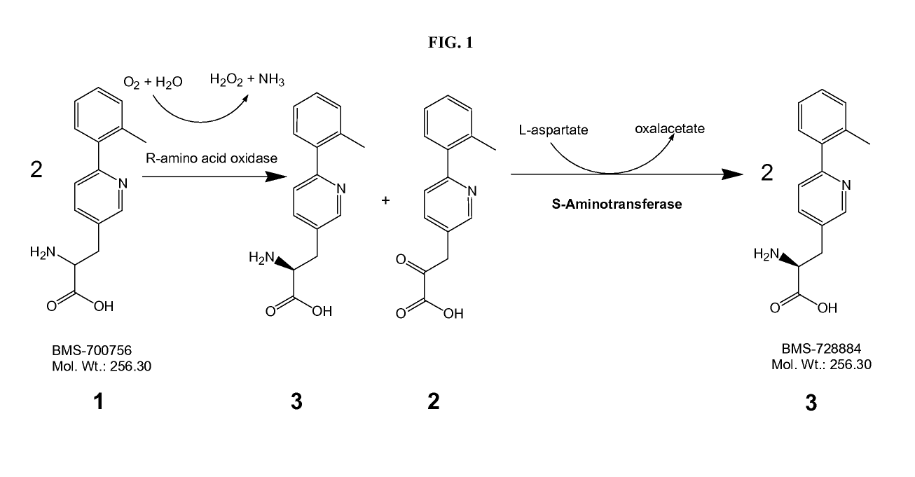

One embodiment disclosed herein is isolated 2-S-amino-3-[3-{6-(2-methylphenyl)}pyridyl]-propionic acid (referred to herein as “BMS-728884”; See FIG. 1, compound 3), or a salt thereof. BMS-728884 is the isolated S enantiomer of racemic amino acid 2-RS-amino-3-[3-{6-(2-methylphenyl)}pyridyl]-propionic acid (referred to herein as “BMS-700756”; See FIG. 1, compound 1).

“R enantiomers” and “S enantiomers” are standard stereochemical definitions known in the art. See, e.g., R. S. Cahn, C. K. Ingold and V. Prelog, Angew. Chem. 78, 413-447 (1966), Angew. Chem. Internat. Ed. Eng. 5, 385-415, 511 (1966); and V. Prelog and G. Helmchen, Angew. Chem. 94, 614-631 (1982), Angew. Chem. Internat. Ed. Eng. 21, 567-583 (1982).

In the context of the compositions disclosed and claimed herein, the term “isolated” means that an (S) enantiomer is isolated from a “R” enantiomer, resulting in no more than 10% “R” enantiomer, and preferably no more than 5%, 4%, 3%, 2%, 1%, or 0.5%, of the “R” enantiomer in the isolated (S) enantiomer.

Enantiomeric purity may be measured using the following method:

Chiral method (a) to determine enantiomeric excess (“ee”): Column: Chirobiotic T 25×0.46 cm; Mobile phase: A. water:methanol 90: 10:05% acetic acid; B. methanol: water 90:10: 0.05% acetic acid; Gradient: 0-3 min, 10% B; 3-15 min, 10-100% B; 15-20 min, 100% B; Flow rate: 1.2 ml/min; Column temperature: 25° C.; Detection: 215 nm; Injection volume: 5 μl; Retention times: S-enantiomer of 1: 14.1 min; R-enantiomer of 1: 14.9 min.

An isolated (S)-enantiomer can be stored in solution, as a powder, or bound to a solid surface.

The isolated (S)-enantiomer can be used, for example, as an intermediate in the preparation of pharmaceuticals, such as GLP-1 mimics or GLP-1 receptor modulators disclosed in WO 2003033671, WO 2004094461, US20040127423, US20030195157, and WO 2003033671. Another embodiment is the use of the isolated BMS-728884, or a salt thereof, for the preparation of intermediates useful in preparing peptide receptor modulators, such as GLP-1 receptor modulators, including agonists or partial agonists. Polypeptide synthesis can be carried out according to standard methods in the art, for example using solid phase, liquid phase, or peptide segment condensation techniques.

The term “salts” refers herein in all aspects and embodiments disclosed herein to the relatively non-toxic, inorganic and organic acid addition salts of compounds of the present invention. These salts can be prepared in situ during the final isolation and purification of the compounds, or by separately reacting the purified compound in its free base form with a suitable organic or inorganic acid and isolating the salt formed. Representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as ammonium, quaternary ammonium, and amine cations including, but not limited to ammonium, tetramethylammonium, tetraethylammonium, methylamine, dimethylamine, trimethylamine, triethylamine, ethylamine, and the like. (See, e.g., Berge S. M. et al., “Pharmaceutical Salts,” J. Pharm. Sci., 1977; 66:1-19 which is incorporated herein by reference.) BMS-728884, or salts thereof, can further be combined with a pharmaceutically acceptable carrier.

Exemplary methods for the synthesis of BMS-728884 are disclosed below.

One embodiment disclosed herein is an acid of BMS-700756 (See FIG. 1, compound 2), or a salt thereof. The acid can be isolated, or can be present in combination with BMS-728884, such as during the synthetic methods described below.

Such isolated acids (e.g., keto acids) may be used, for example, as intermediates in the synthesis of BMS-728884. Thus, the present invention further provides for the use of the keto acid of BMS-700756, or a salt thereof, for the synthesis of BMS-728884. Such methods are described in more detail below. The keto acid, or salts thereof, can further be combined with a pharmaceutically acceptable carrier. The isolated keto acid can be stored in solution, as a powder, or bound to a solid surface.

Exemplary methods for synthesis of the keto acid of BMS-700756 are described below.

An additional embodiment is a purified polypeptide (and methods for purifying such polypeptides), comprising or consisting of an amino acid sequence of SEQ ID NO:2 or SEQ ID NO:4, or functional equivalents thereof. Methods for purifying the polypeptides are described in detail herein.

SEQ ID NO:2 is an aminotransferase (adenylmethionine-8-amino-7-oxononanoate aminotransferase (AAOA) Burkholderia species. SEQ ID NO:4 is an amino acid dehydrogenase from Sporosarcina ureae. A detailed description of the polypeptides is provided in the examples.

The term “polypeptide” as used herein, in its broadest sense, refers to a sequence of subunit amino acids, amino acid analogs (both naturally occurring and synthetic), or peptidomimetics. The subunits are linked by amide bonds.

The polypeptides of the invention can be used, for example, in the conversion of racemic mixtures of (R,S) amino acids to the (S) enantiomer, as disclosed herein. The polypeptides can be present in solution, lyophilized as a powder, or present on a solid support, such as a chromatography or other column, membranes, polymers, and beads.

As used herein, the term “substantially purified” means that the active polypeptide has been separated from its in vivo cellular environment, and from other contaminating compounds used in its purification. Thus, the polypeptide can either be purified from: natural sources, recombinant polypeptide can be purified from the transfected host cells as disclosed below, or can be chemically synthesized using standard techniques, including but not limited to solid phase, liquid phase, or peptide condensation techniques, or any combination thereof.

In a preferred embodiment, the polypeptides are produced by transfected cells such as those disclosed below, and purified and described herein. (See, e.g., Molecular Cloning: A Laboratory Manual Sambrook, et al., 1989, Cold Spring Harbor Laboratory Press.) The polypeptides can be purified from prokaryotic or eukaryotic sources. In various further preferred embodiments, the polypeptides are purified from recombinant bacterial cells, including but not limited to E. coli.

Another embodiment includes an isolated nucleic acid comprising or consisting of a nucleic acid that encodes a polypeptide of SEQ ID NO:2 or SEQ ID NO:4, or functional equivalents thereof. Such nucleic acids will be readily apparent to those of skill in the art, given the disclosed polypeptide sequences of SEQ ID NOS: 2 and 4. In a preferred embodiment, the nucleic acid comprises, or consists of, the nucleic acid of SEQ ID NO: 1 or SEQ ID NO:3.

The term “isolated” with respect to the nucleic and amino acid embodiments disclosed and claimed herein, means that a nucleic acid is removed from an in vivo environment, and is separated from other nucleic acids from which it was derived.

The isolated nucleic acid may comprise DNA, RNA, cDNA, or a genomic clone. The isolated nucleic acid may further comprise additional residues useful for promoting expression and/or purification of the encoded polypeptide, including but not limited to polyA sequences, modified Kozak sequences, and sequences encoding epitope tags, export signals, secretory signals, and localization signals.

The isolated nucleic acids can be isolated from naturally occurring sources using standard techniques in the art, or can be prepared using standard nucleic acid synthesis techniques.

The nucleic acids are useful, for example, for preparing recombinant expression vectors to synthesize the polypeptide(s) encoded by the isolated nucleic acids. Thus, a related embodiment is recombinant expression vectors comprising an isolated nucleic acid of the invention. As used herein, a “recombinant expression vector” includes any vector that operatively links a nucleic acid coding region or gene to any promoter capable of effecting expression of the gene product. The promoter sequence used to drive expression of the nucleic acids may be constitutive or inducible. The construction of expression vectors for use in transfecting prokaryotic or eukaryotic cells is well known in the art, and thus can be accomplished via standard techniques. (See, e.g., Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.).

The expression vector must be replicable in the host organisms either as an episome or by integration into host chromosomal DNA. In a preferred embodiment, the expression vector comprises a plasmid. However, other expression vectors that serve equivalent functions, such as viral vectors, would be suitable for use.

Further embodiments include recombinant host cells transfected with a recombinant expression vector comprising a nucleic acid that encodes the polypeptide of SEQ ID NO:2 and/or SEQ ID NO:4. Thus, the recombinant host cells may comprise a recombinant expression vector further comprising a nucleic acid that encodes the polypeptide of SEQ ID NO:2; a recombinant expression vector comprising a nucleic acid that encodes the polypeptide of SEQ ID NO:4; a first recombinant expression vector comprising a nucleic acid that encodes the polypeptide of SEQ ID NO:2 and a second recombinant expression vector comprising a nucleic acid that encodes the polypeptide of SEQ ID NO:4, or a single recombinant expression vector comprising nucleic acids that encode the polypeptide of SEQ ID NO:2 and SEQ ID NO:4. The host cells can be either prokaryotic or eukaryotic, and the cells can be transiently or stably transfected.

In a preferred embodiment, the host cells are prokaryotic, and in a more preferred embodiment, the host cells are E. coli. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, e.g., Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). Such host cells are useful, for example, for producing the polypeptides of SEQ ID NO:2 and SEQ ID NO:4, which can be used in the methods disclosed below. As shown in the examples below, these polypeptides can be used in enzymatic assays either as purified proteins, or when present in cell extracts derived from the recombinant host cells of the invention.

Another embodiment is a cell extract derived from a recombinant host cell. As used herein, the term “cell extract” refers to a polypeptide-enriched fraction derived from recombinant host cells that may include polypeptides such as the polypeptides of SEQ ID NO:2 and/or SEQ ID NO:4 present in an enzymatically active form. Such extracts can be prepared by any method known in the art for making such cell extracts, including but not limited to cell fractionation, partial purification of the relevant enzymatic activity, clarification by addition of polyethyleneamine (“PEI”) to the extract and centrifugation, and the methods disclosed in the examples below. Thus, embodiments include cell extracts comprising the polypeptide of SEQ ID NO:2, cell extracts comprising the polypeptide of SEQ ID NO:4, and cell extracts comprising the polypeptide of SEQ ID NO:2 and SEQ ID NO:4. Such cell extracts comprising the polypeptides of SEQ ID NO:2 and SEQ ID NO:4 can be made by combining extracts from different recombinant host cells, or can be made by preparing a cell extract from a recombinant host cell transfected with one or more recombinant expression vectors directing expression of the polypeptide of SEQ ID NO:2 and SEQ ID NO:4.

As noted above, the cell extracts can be used, for example, to carry out the enzymatic reactions of the invention disclosed herein.

Further described herein are methods for converting a racemic amino acid to an (S) amino acid, comprising (a) reacting a racemic amino acid with an amount of an amino acid oxidase (e.g., R-amino acid oxidase) to produce a mixture of (S) amino acid and keto acid; and (b) reacting the mixture with (i) an amount of an aminotransferase of SEQ ID NO:2 in the presence of an amino acid, or (ii) an amino acid dehydrogenase of SEQ ID NO:4 in the presence of an amine donor group (e.g. ammonia or ammonium ion) and a cofactor selected from the group consisting of NADH; NAD+formate+formate dehydrogenase; and NAD+glucose+glucose dehydrogenase, to produce the (S) amino acid.

The amino acid oxidase may be any capable of converting an (R,S) racemic mixture to a mixture of the (S) enantiomer and a keto acid, including, but not limited to, D-amino acid oxidase from Trigonopsis variabilis, and D-amino oxidase from porcine kidney (Shah et al., Tetrahedron Letters (1994), 35(1), 29-32; Hanson et al., Bioorganic & Medicinal Chemistry (1999), 7(10), 2247-2252.). The amino acid oxidase is preferably an R-amino acid oxidase.

The aminotransferase of SEQ ID NO:2, and the amino acid dehydrogenase of SEQ ID NO:4, are disclosed in detail herein. In one embodiment, the aminotransferase or amino acid dehydrogenase is present in a cell extract of the invention. In another embodiment, the aminotransferase or amino acid dehydrogenase is substantially purified, as disclosed herein.

As used herein, an amino acid is an organic compound containing an amino group, a carboxylic acid group, and any of various side groups, and includes naturally occurring amino acids and non-naturally occurring amino acids.

As used herein a “racemic” amino acid is one that contains approximately equal amounts of the R and S enantiomer of the amino acid.

As used herein, an “amino donor” includes any compound capable of donating an amino group, including, but not limited to, amine-containing amino acids such as aspartic acid, glutamic acid or salts thereof, ammonia and salts thereof, and aliphatic or aromatic amines and salts thereof.

As used in the context of resolving (S) amino acids, the term “converting” means to convert the racemic (R,S) amino acid to a mixture of (S) amino acid and keto acid, with little or no (R) enantiomer of the amino acid remaining. In various preferred embodiments, converting yields less than 10%, of (R) enantiomer remaining, and more preferably less than 5%, 4%, 3%, 2%, 1%, or 0.5%, of (R) enantiomer remaining.

Enantiomeric purity is measured as defined above.

In one embodiment, the amino acid is a compound of Formula (II): 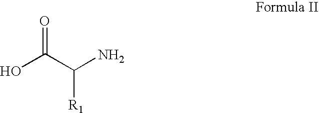
wherein R₁ is selected from the group consisting of alkenyl, alkyl, alkynyl, aryl, arylalkyl, cycloalkyl, cycloalkylalkyl, heteroaryl, and heteroarylalkyl. In a preferred embodiment, R₁ is heteroarylalkyl wherein the heteroaryl is pyridinyl optionally substituted with 2-methylphenyl. In a most preferred embodiment, the amino acid is BMS-700756, or a salt thereof, and the (S) enantiomer is BMS-728884, or a salt thereof.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means phenyl or naphthyl group. The aryl groups of the present invention are optionally substituted with 1, 2, 3, 4, or 5 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂, (NZ₁Z₂) carbonyl, and a phenyl group optionally substituted with 1, 2, 3, 4, or 5 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, nitro, —NZ₁Z₂, and (NZ₁Z₂)carbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “carboxy” as used herein, means a —CO₂H group.

The term “cyano” as used herein, means a —CN group.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The cycloalkyl groups of the present invention are optionally substituted with 1, 2, 3, or 4 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, oxo, —NZ₁Z₂, (NZ₁Z₂) carbonyl, and a phenyl group optionally substituted with 1, 2, 3, 4, or 5 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, nitro, —NZ₁Z₂, and (NZ₁Z₂)carbonyl.

The term “cycloalkylalkyl” as used herein, means a cycloalkyl group, as defined herein, appended to the parent molecular moiety through an alkyl group, as defined herein.

Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring. The 5 membered ring may consist of two double bonds and one, two, three or four nitrogen atoms and optionally one oxygen or sulfur atom. The 6 membered ring may consist of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The heteroaryl groups of the present invention are optionally substituted with 1, 2, 3, or 4 substituents independently selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, nitro, —NZ₁Z₂, (NZ₁Z₂)carbonyl, and a phenyl group optionally substituted with 1, 2, 3, 4, or 5 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, nitro, —NZ₁Z₂, and (NZ₁Z₂)carbonyl.

The term “heteroarylalkyl” as used herein, means a heteroaryl, as defined herein, appended to the parent molecular moiety through an alkyl group, as defined herein. Representative examples of heteroarylalkyl include, but are not limited to pyridinylmethyl.

The term “heterocycle” or “heterocyclic” as used herein, means a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. Representative examples of heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The heterocycles of this invention are optionally substituted with 1, 2, or 3 substituents independently selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, oxo, —NZ₁Z₂, (NZ₁Z₂)carbonyl, and a phenyl group optionally substituted with 1, 2, 3, 4, or 5 substituents selected from the group consisting of alkenyl, alkoxy, alkoxycarbonyl, alkyl, alkylcarbonyl, alkylthio, alkynyl, carboxy, cyano, formyl, haloalkoxy, haloalkyl, halogen, hydroxy, hydroxyalkyl, mercapto, nitro, —NZ₁Z₂, and (NZ₁Z₂)carbonyl.

The term “heterocyclealkyl” as used herein, means a heterocycle, as defined herein, appended to the parent molecular moiety through an alkyl group, as defined herein.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “NZ₁Z₂” as used herein, means two groups, Z₁ and Z₂, which are appended to the parent molecular moiety through a nitrogen atom. Z₁ and Z₂ are each independently hydrogen, alkyl, alkylcarbonyl, or formyl. Representative examples of NZ₁Z₂ include, but are not limited to, amino, methylamino, acetylamino, and acetylmethylamino.

The term “(NZ₁Z₂) carbonyl” as used herein, means a NZ₁Z₂ group, as defined herein, appended to the parent molecular moiety through a carbonyl group, as defined herein. Representative examples of (NZ₁Z₂)carbonyl include, but are not limited to, aminocarbonyl, (methylamino)carbonyl, (dimethylamino)carbonyl, and (ethylmethylamino)carbonyl.

Representative reaction conditions for carrying out the methods described and claimed herein are described below. As will be understood by those of skill in the art, the reaction conditions can be modified using standard techniques, in light of the teachings provided herein.

In a preferred embodiment the reactions are carried out under the following conditions: in an aqueous solvent; at a pH of between 7 and 8.5; with agitation of between 200-500 rpm, more preferably between 250 rpm and 400 rpm; for between 2 and 25 hours; and at a temperature between about 20° C. and about 50° C.; more preferably between 20° C. and 32° C., more preferably at about 30° C., or at room temperature.

Converting racemic amino acids to an (S) amino acid, comprises: (a) reacting a racemic amino acid with an amount of an amino acid oxidase sufficient to produce a mixture of (S) amino acid and keto acid; and (b) reacting the mixture with (i) an amount of an aminotransferase of SEQ ID NO:2 in the presence of an amino acid, or (ii) an amino acid dehydrogenase of SEQ ID NO:4, in the presence of an amino donor group and a cofactor selected from the group consisting of NADH; NAD+formate+formate dehydrogenase; and NAD+glucose+glucose dehydrogenase, to produce the (S) amino acid. For this reaction, NADH or NADH generating system (NAD, formate and formate dehydrogenase or NAD, glucose and glucose dehydrogenase) is used.

An additional embodiment is a method for converting a keto acid to an (S) amino acid, comprising reacting a keto acid of general formula I: 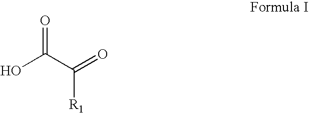
with (i) an amount of an aminotransferase of SEQ ID NO:2 in the presence of an amino acid, or (ii) an amino acid dehydrogenase of SEQ ID NO:4 in the presence of an amino donor and a cofactor selected from the group consisting of NADH; NAD+formate+formate dehydrogenase; and NAD+glucose+glucose dehydrogenase, to produce an (S) amino acid, wherein R₁ is selected from the group consisting of alkenyl, alkyl, alkynyl, aryl, arylalkyl, cycloalkyl, cycloalkylalkyl, heteroaryl, and heteroarylalkyl.

In a preferred embodiment, R₁ is heteroarylalkyl wherein the heteroaryl is pyridinyl optionally substituted with 2-methylphenyl. These terms for R₁ are as defined above.

The aminotransferase of SEQ ID NO:2 and the amino acid dehydrogenase of SEQ ID NO:4 are disclosed in detail above. In one embodiment, the aminotransferase or amino acid dehydrogenase are present in a cell extract of the invention. In another embodiment, the aminotransferase or amino acid dehydrogenase are substantially purified, as disclosed herein.

As used in the context of a method for converting a keto acid to an (S) amino acid, comprising reacting a keto acid of general formula I, the term “converting” means to convert the keto acid to the (S) amino acid, with little or no keto acid remaining. In various preferred embodiments, converting results in no more than 10% of keto acid remaining, and more preferably no more than 5%, 4%, 3%, 2%, or 1%, keto acid remaining.

In a preferred embodiment, the keto acid is compound 2 in FIG. 1, or a salt thereof.

An additional embodiment is a method for converting 2-RS-amino-3-[3-{6-(2-methylphenyl)}pyridyl]-propionic acid to S-Amino-[3-{6-(2-methylphenyl)}pyridyl]-propionic acid, comprising reacting 2-RS-amino-3-[3-{6-(2-methylphenyl)}pyridyl]-propionic acid, or a salt thereof, with an amino acid oxidase in combination with a non-enzymatic reducing agent in the presence of an inorganic catalyst, to produce S-Amino-[3-{6-(2-methylphenyl)}pyridyl]-propionic acid.

In this aspect, the amino acid oxidase enzyme reacts only with the R-amino acid (which is 50% of the racemic amino acid), and leaves the S-amino acid unchanged. The R-amino acid is oxidized by the amino acid oxidase enzyme, forming an imino acid-enzyme complex. The imino acid in the complex is reduced by the inorganic catalyst to RS-amino acid. Subsequently, the S-amino acid portion of the RS-amino acid remains unchanged, while the R-amino acid portion is oxidized by the enzyme. Ultimately, the S-amino acid is highly enriched, with negligible amounts of R-amino acid present.

In a preferred embodiment the inorganic catalyst is sodium borohydride, ammonia-borane, palladium, or ammonium formate, or salts thereof. In a preferred embodiment, the non-enzymatic reducing agent comprises ammonia, borohydride, borane-ammonia, or a borane-amine complex, or a salt thereof. In a more preferred embodiment, the non-enzymatic reducing agent comprises ammonia, or salt thereof, and is used in combination with a metal catalyst.

### EXAMPLES

The following Examples illustrate embodiments of the present invention, and are not intended to limit the scope of the attached claims.

### Example 1

Cloning and Expression of the Burkholderia Sp (S)-Aminotransferase Gene Purification of Native (S)-Aminotransferase

A Burkholderia species was isolated from soil, and the strain was identified by ribosomal 16S RNA technique. This strain was used for the purification of adenylmethionine-8-amino-7-oxononanoate aminotransferase (AAOA).

For preparation of crude cell-free extracts, 25 g (wet weight) of cells was suspended in 300 ml of 50 mM potassium phosphate buffer pH 7.0 containing 1 mM DTT and 25 μM pyridoxal 5′-phosphate (PLP) (buffer A), then disintegrated with a Microfluidizer at 12,000 psi for 3 times. The disintegrated cells were centrifuged at 45,000×g for 90 min at 4° C. to remove the cell debris. The supernatant was collected and stored at −80° C. for subsequent enzyme purification. Native (S)-aminotransferase was purified to homogeneity by a 4-step procedure as follows:

a. Butyl-sepharose: Crude extracts (65 ml, 345 mg of protein) containing 0.5 M ammonium sulfate were loaded on 20 ml (2×15 cm) butyl-sepharose 4 Fast Flow (Sigma) column equilibrated with 150 ml of buffer A containing 0.5 M ammonium sulfate. The enzyme was eluted with 100 ml of ammonium sulfate with a linear gradient (0.5 M to 0) at a flow rate of 1 ml/min, and 3 ml fractions were collected. The active fractions (21 ml) were combined and concentrated through a PM-30 membrane to approximately 3 ml, which was diluted to 20 ml with 10 mM potassium phosphate pH 7.0 containing 1 mM DTT and 5 mM magnesium sulfate (buffer B).

b. Red-120™ Affinity: The 20 ml fraction was applied onto a Red-120 dye (Sigma) affinity column (18 ml) equilibrated with 150 ml of buffer B, and the flow-through was collected. The column was then washed with 25 ml of buffer B and collected. The flow-through and wash fractions (45 ml) were combined and concentrated through a PM-30 membrane to approximately 2 ml.

c. UnoQ™ FPLC: The concentrated enzyme solution was injected to a UnoQ column (1×3.8 cm, Bio-Rad) equilibrated with 24 ml of 25 mM potassium phosphate pH 7.0 containing 1 mM DTT and 0.1 M NaCl at flow rate of 1 ml/min. After washing with 12 ml of the buffer, the column was eluted with 16 ml of linear gradient (0.1 to 0.2 M NaCl), and 0.5 ml fractions were collected. The enzyme activity was in the unbound fractions, and the active fractions (8 ml) were combined and concentrated to approximately 0.5 ml.

d. Superdex™ 200 FPLC: The enzyme solution was injected to a Superdex column (1.2×30 cm, Pharmacia) equilibrated with 23 ml of 25 mM potassium phosphate pH 7.0 containing 1 mM DTT and 100 mM NaCl at flow rate of 0.5 ml/min. The enzyme was eluted with 23 ml of 25 mM potassium phosphate pH 7.0 containing 1 mM DTT and 0.1 M NaCl, and 0.5 ml fractions were collected. The active fractions (1.5 ml) were combined and concentrated to 0.2 ml for subsequent analyses.

TABLE 1

| | Total | Total | Specific | |
|---|---|---|---|---|
| | Protein | Activity | Activity | Purification |
| Step | (mg) | (μg/h) | (μg/h/mg) | (fold) |
| Crude Extract | 345.0 | 123,383 | 330.1 | 1 |
| Butyl-Sepharose | 42.6 | 98,004 | 2,300.6 | 7.0 |
| Red-120 | 3.65 | 34,546 | 9,470.4 | 28.7 |
| UnoQ | 0.56 | 30,556 | 48,857.1 | 148.0 |
| Superdex 200 | 0.024 | 13,327 | 111,062.5 | 336.3 |

An enzyme from Burkholderia sp. that catalyzed the transamination reaction shown in FIG. 1 was purified to homogeneity as described herein. A sample of the protein was submitted for partial amino acid sequencing following tryptic digestion. Four sequences were obtained:

1) APALPHATQA (N-terminus)

2) KADGVYLWDSDGN

3) KELADAAYR

4) DEGIVER

A BLAST2 homology search using the above sequences showed regions of homology to adenosylmethionine-8-amino-7-oxononanoate aminotransferases (AAOA) from Burkholderia cenocepacia and Borrelia cepacia. For use in polymerase chain reaction (PCR), oligonucleotide primers were prepared based on the corresponding codons of the amino acids. The direction of the primers (i.e., sense and/or anti-sense) were determined using the likely location of the amino acid sequence within the protein as compared to similar aminotransferases.

TABLE 2

| | | Corresponds to |
|---|---|---|
| Primer name | Sequence | (direction) |
| Oligo 557 | GCICCICAYGCIACICARGC | APHATQA |
| | (SEQ ID NO: 5) | (sense) |
| Oligo 558 | TAIGCIGCRTCIGCIGRYTC | ELADAYY |
| | (SEQ ID NO: 6) | (anti-sense) |
| Oligo 559 | AARGCIGAYGGIGTITAYYCITGGGA | KADGVYLWD |
| | (SEQ ID NO: 7) | (sense) |
| Oligo 560 | AARGARYCIGCIGAYGCIGCITA | KELADAAY |
| | (SEQ ID NO: 8) | (sense) |
| Oligo 561 | CRYTCIACDATICCYTCRTC | DEGIFVER |
| | (SEQ ID NO: 9) | (anti-sense) |

Standard nucleoside bases are indicated by “A”, “T”, “C”, or “G”. “I”=inosine (replaces all four bases). Mixed bases are: “R” (A+G); “S” (C+G); “W” (A+T); “Y” (C+T).

Oligo pairs 557+561 and 560+561 were used with the FailSafe™ series of PCR buffer (Epicentre Technologies) and Burkholderia sp. chromosomal DNA as template. The PCR (10 μL final volume) was carried out in a Hybaid™ PCR Express thermocycler with the following parameters:

TABLE 3

| Stage 1 | Stage 2 | Stage 3 | Stage 4 | Stage 5 |
|---|---|---|---|---|
| 94° 1 min | 94° 30 sec | 94° 30 sec | 94° 30 sec | 72° 5 min |
| | 55° 30 sec | 55°-->40° 30 sec | 40° 30 sec | |
| | 72° 30 sec | 72° 30 sec | 72° 30 sec | |
| 1 cycle | 4 cycles | 20 cycles | 5 cycles | |

A fragment of the expected size (˜1056 bp based on comparison to known AAOA genes) was obtained using oligos 557+561. The best amplification was observed in buffer “D.” The reaction was scaled up 40-fold and included: 2× FailSafe buffer “D”, 200 μL; 10× loading dye, 40 μL; oligonucleotide, 1.6 μL each; Z-Taq DNA polymerase (PanVera), 2.0 μL; Burkholderia sp. DNA (1 mg/mL), 2.0 μL; dH₂O, 152.8 μL. The entire reaction mix was electrophoresed on a 1.0% agarose gel for 2 hr at 100 v in TAE buffer (0.04 M Trizma base, 0.02 M acetic acid, and 0.001 M EDTA, pH 8.3) containing 0.5 μg/ml ethidium bromide. The fragment was excised from the gel and purified using a Qiagen Gel Purification Kit (Qiagen). To verify this fragment represented the AAOA gene, additional PCR reactions were conducted as above using oligo pairs 557+558, 558+559, 559+561, and 560+561, using the amplified fragment as template. In two instances (oligos 557+558 and 558+559), a fragment of the expected size (˜257 and 112 bp, respectively) was obtained.

To isolate the entire gene, Burkholderia sp. DNA was first cleaved with a series of 9 restriction endonucleases (BamHI, BglII, EcoRI, EcoRV, and HindIII, KpnI, NotI, PstI, and SpeI). Reactions contained 5 μg DNA, appropriate buffer, and units enzyme in 30 μL final volume. Digests were carried out for 3 hr at 37° C., then electrophoresed in a 0.8% TAE-agarose gel at 16 v for 18 hr. The DNA was transferred to Hybond™ N+ nylon filters under alkaline conditions using the VacuGene™ vacuum blotting unit (Amersham-Pharmacia). Several attempts to prepare a Burkholderia sp. AAOA-specific DNA probe using the DIG PCR Probe Synthesis kit (Roche Biochemicals) using the recommended conditions by the manufacturer were unsuccessful. This may have been due to the use of mixed primers in the reaction. As an alternative, a probe was prepared using specific primers based on the known DNA sequence of the AAOA gene from Burkholderia cepacia.

TABLE 4

| | | |
|---|---|---|
| Oligo | GGGCTTCAACCACTTCTTCTAT | bp 387-412 of |
| 564 | TGC | Burkholderia cepacia |
| | (SEQ ID NO: 10) | AAOA (sense) |
| Oligo | GCCGAAGTGCTGGTGTGCGAA | bp 898-875 of |
| 565 | CACC | Burkholderia cepacia |
| | (SEQ ID NO: 11) | AAOA (anti-sense) |

A labeled fragment was obtained as indicated by an increase in molecular weight compared to unlabeled DNA due to incorporation of dUTP-digoxigenin. Hybridization to the blotted DNA digests was performed in EasyHyb solution (Roche). Stringent wash was 0.5×SSC (20×SSC=173.5 g NaCl and 88.2 g NaCl, pH 7.0), 0.1% sodium dodecyl sulfate at 68° C. for 2×15 minutes. A single hybridizing BglII fragment of ˜3800 base pairs was obtained and used for mini-library preparation.

Twenty-five μg of chromosomal DNA was cleaved with 100 U BglII in a total volume of 100 μL (37° C., 3 hr) and electrophoresed as described above. The region from ˜3200-4200 base pairs was cut from the gel and the DNA purified using QIAquick™. The isolated DNA was ligated to pZero2 vector DNA digested with BamHI (the overhanging nucleotides are compatible with those of BglII) at a 5:1 (insert:vector) molar ratio in a total volume of 10 μl at 22° C. for 2 hr. DNA was precipitated by addition of 15 μl dH₂O and 250 μL 1-butanol, and pelleted at 13,000×g in a microcentrifuge for 5 min. Liquid was removed by aspiration, and the DNA was dried in a SpeedVac™ (Savant Instruments, Farmingdale, N.Y.) for 5 min under low heat. The pellet was resuspended in 5 μl dH₂O. The resuspended DNA was transformed by electroporation into 0.04 ml E. coli DH10B competent cells (Invitrogen). SOC medium was immediately added (0.96 ml; SOC=0.5% yeast extract, 2% tryptone, 10 mM NaCl, 2.5 mM KCl, 10 mM MgCl₂, 10 mM MgSO₄, and mM glucose per liter), and the cells incubated in a shaker for 1 hr at 37° C. and 225 rpm. Colonies containing recombinant plasmids were selected on LB agar plates containing 50 μg/ml kanamycin sulfate (Sigma Chemicals, St. Louis, Mo.). Sufficient cells to give about 15,000 colonies were spread onto a 132 mm Hybond N+ membrane (Amersham Pharmacia) placed on top of LB agar medium containing 50 μg/ml kanamycin and incubated at 37° C. for 20 hr.

Colonies were replicated onto two fresh filters placed on top of LB kanamycin agar medium. The filters were incubated at 37° C. for 4 hr. Colonies were lysed in situ by placing the filters on a piece of Whatman 3MM paper (Whatman International, Maidstone, UK) saturated with 0.5 M NaOH for 5 min. The filters were dried for 5 min on Whatman paper, then neutralized on 3MM paper soaked in 1.0 M Tris-HCl, pH 7.5 for 2 min, and dried for 2 min. Membranes were placed on top of 3MM paper saturated with 1.0 M Tris-HCl, pH7.0/1.5 M NaCl for 10 min. DNA was crosslinked to the filters by exposure to ultraviolet light in a Stratagene UV Stratalinker™ 2400 set to “auto crosslink” mode (Stratagene, La Jolla, Calif.). Cell debris was removed from the membranes by immersing in 3×SSC/0.1% SDS and wiping the surface with a wetted Kimwipe® (Kimberly-Clark Co., Roswell, Ga.), then incubating in the same solution heated to 65° C. for 3 hr with agitation. Filters were rinsed with dH₂O and used immediately or wrapped in SaranWrap® and stored at 4° C. Hybridization, washing, and detection of the colony blots were performed as described above using the labeled PCR probe. About 100 strongly hybridizing colonies were obtained; six were removed from the master plate, inoculated into LB-kanamycin liquid medium, and grown at 37° C. for 24 hr, 250 rpm. Plasmid DNA was isolated using the Fast Plasmid™ DNA kit from Eppendorf. Restriction digests with KpnI+Not I (these restriction sites surrounds the cloned fragment) and PstI indicated 5/6 samples contained a about 4.0 kb insert and that these were identical to each other. PCR using the five positive plasmids, a template, and oligos 564+565 gave strong amplification of the expected 600-bp fragments. One plasmid was chosen for further study and named “pZerO2-AAOA.”

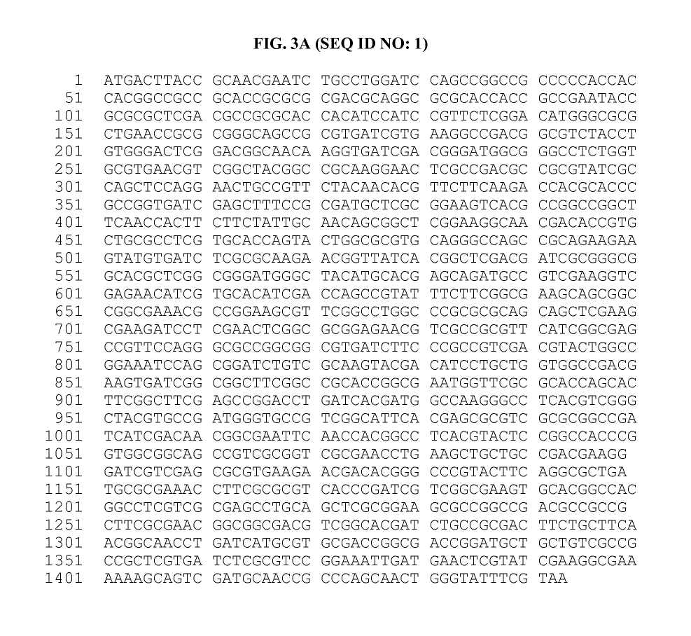

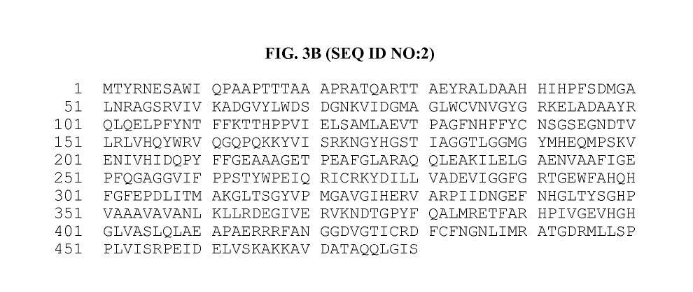

For rapid DNA sequencing of the insert in pZerO-AAOA, primer sites were introduced at random using the New England Biolabs Genome Priming System kit. Colonies containing transposons in the plasmid were identified by selection on LB agar medium+kanamycin and chloramphenicol at 20 and 15 μg/mL, respectively. Colonies were grown, plasmid DNA extracted, and sequencing performed. The AAOA gene was identified within the 4000-base pair insert (SEQ ID NO:1; see FIG. 3A).

Expression Cloning of the AAOA Gene:

Primers were prepared for amplification of the AAOA for ligation to pBMS2000 and pBMS2000-PPFDH-ALD expression vectors:

TABLE 5

| | | |
|---|---|---|
| Primer | | Corresponds to |
| name | Sequence | (direction) |

| | | |
|---|---|---|
| Oligo | GACATATTTAAATCATATGACT | SwaI/NdeI/5′ |
| 569 | TACCGCAACGAATCTGCC | end Burkholderia sp. |
| | (SEQ ID NO: 12) | AAOA (sense) |
| Oligo | GATTTAAATCCCGGGTTACGAA | SwaI/SmaI/stop 3′ |
| 570 | ATACCCAGTTGCTGGGCGG | end Burkholderia sp. |
| | (SEQ ID NO: 13) | AAOA gene |
| | | (anti-sense) |

The FailSafe series of buffers were tested using oligos 569+570 with pZerO2-AAOA as template for PCR. Buffers “D” and “F’ both gave strong amplification of the expected ˜1450-bp fragment; the former buffer was used to scale up both reactions 20-fold. A MinElute column was used to purify the fragments after addition of 5 vol buffer PB before loading onto the column.

The PCR reaction with oligos 569+570 (about 2 μg) was cleaved with 10 U each NdeI and SmaI in a 40 μL volume (37° C., 2 hr). Samples were electrophoresed and purified after excision from the agarose gel using the MinElute™ kit. The digested PCR using oligos 569+570 (100 ng) was ligated to 30 ng NdeI-SmaI-cut pBMS2000 using the Fast Link kit as previously described.

Precipitation, recovery of DNA, and transformation of DH10B were carried out. Eighteen Km^{r} colonies from each experiment were tested for presence of insert using the appropriate primer set. In both instances, one colony supported amplification of the correct fragment. Plasmid DNA was prepared from both samples and cleaved with NdeI+SmaI and EcoRI (pBMS2000-AAOA). The molecular weight of protein is 54,000 Daltons.

Growth of Recombinant E. coli:

MT5-M2 contained 2.0% Hy-Pea (Quest International), 1.85% Tastone-154 (Sensient Bionutrients), 0.6% Na₂HPO₄, 0.125% (NH₄)₂SO₄, and 4.0% glycerol, adjusted to pH 7.2 pre-sterilization. Using a 50 mg/mL filter-sterilized aqueous stock solution, kanamycin sulfate was aseptically added to a level of 50 μg/mL after autoclaving. For shake flask expression work, cells were initially grown in MT5-M2 for 20-24 hr at 30° C. and 250 rpm. The optical density at 600 nm (OD₆₀₀) was recorded and fresh medium was inoculated with the culture to a starting OD₆₀₀ of 0.30. The flask was incubated as described above until the OD₆₀₀ reached ˜0.8-1.0. Isopropyl-thio-β-D-galactoside (IPTG) was added from a 1M filter-sterilized stock in de-ionized H₂O to the desired final concentration (50 μM or 1 mM) and the culture was allowed to grow for varying lengths of time before harvesting by centrifugation.

Recombinant (S)-Aminotransferase:

Strain SC16541 [E coli BL21(pBMS2000-AAOA)] was used for the production of adenylmethionine-8-amino-7-oxononanoate aminotransferase (AAOA) from the Burkholderia sp strain.

Preparation of Cell Free Extract:

For preparation of crude cell-free extracts, 25 g (wet weight) of cells was suspended in 300 ml of 50 mM pH 7.0 potassium phosphate buffer containing 1 mM DTT and 25 μM PLP (buffer A), then disintegrated with a Microfluidizer at 12,000 psi for 3 passes. The disintegrated cells were centrifuged at 45,000×g for 90 min at 4° C. to remove the cell debris. The supernatant was collected and stored at −80° C. for enzyme purification and other studies.

### Example 2

### Cloning and Expression of the Sporosarcina ureae Amino Acid Dehydrogenase Gene

Purification of Amino Acid Dehydrogenase from Sporosarcina ureae SC16048:

Native amino acid dehydrogenase from Sporosarcina ureae SC16048 was purified by a 6 step protocol. For preparation of crude cell-free extracts, 80 g (wet weight) of cells was suspended in 320 ml of 50 mM potassium phosphate buffer pH 7.0 containing 1 mM DTT, then disintegrated with a Microfluidizer at 12,000 psi for 3 times. The disintegrated cells were centrifuged at 45,000×g for 90 min at 4° C. to remove the cell debris, and approximately 250 ml of cell-free extract was obtained and stored at −20° C. for enzyme purification.

To 27 ml of cell-free extract, 4.75 g of solid ammonium sulfate was added slowly with stirring to 30% saturation. The solution was then centrifuged at 50,000×g for 60 min to remove the precipitated proteins. The supernatant after the centrifugation was applied to a butyl-sepharose column (25 ml, 1.5×25 cm) equilibrated with 50 mM potassium phosphate buffer pH 7.0 containing 1 mM DTT and 1 M ammonium sulfate.

After washing with 50 ml of the same buffer, the enzyme was eluted with 100 ml of ammonium sulfate with a linear gradient (1 M to 0) at flow rate of 1 ml/min, and 4 ml fractions were collected. The active fractions were combined and concentrated to 5 ml. This concentrate was applied onto 20 ml of Heparin-agarose in a column (Type I, Sigma) equilibrated with 200 ml of 10 mM potassium phosphate buffer pH 7.0 containing 0.5 mM DTT. The column washed with 20 ml of the same buffer. The flow-through and wash fractions were combined and concentrated to approximately 2 ml for the first UnoQ-FPLC column. The first UnoQ column (1×3.8 cm, Bio-Rad) was equilibrated with 24 ml of 10 mM potassium phosphate pH 7.0 containing 0.5 mM DTT at flow rate of 1 ml/min. After injecting the 2 ml sample and washing with 12 ml of the buffer, the column was eluted with 16 ml of linear gradient (0 to 0.3 M NaCl), and 1 ml fractions were collected. The active fractions (9 ml) were combined and concentrated to 2 ml for a second UnoQ-FPLC column.

The concentrate after desalting was injected to a second UnoQ-FPLC that was equilibrated with 24 ml of 10 mM potassium phosphate pH 7.0 containing 0.5 mM DTT and 0.1 M NaCl at a flow rate of 1 ml/min. After washing with 12 ml of the equilibration buffer, the column was eluted with 16 ml of linear gradient (0.1-0.25 M NaCl), and 1 ml fractions were collected. The active fractions (7 ml) were combined and concentrated to 2 ml for the final chromatography. The purification of the amino acid dehydrogenase was achieved by a third UnoQ column. The column was equilibrated with 24 ml of 10 mM potassium phosphate pH 7.0 containing 0.5 mM DTT and 0.175 M NaCl at a flow rate of 1 ml/min. The concentrate from the previous column (2 ml) was injected onto the column after washing with 12 ml of the equilibration buffer. Then, the column was eluted with 16 ml of linear gradient (0.175-0.3 M NaCl), and 0.75 ml fractions were collected. The amino acid dehydrogenase was purified to homogeneity by this 6-step procedure. The protein band at 42 kDa on SDS-PAGE corresponding to the dehydrogenase activity was blotted onto PVDF membranes for protein sequencing. The purification scheme for the amino acid dehydrogenase is summarized in the following table.

TABLE 6

| | Total | Total | Specific | |
|---|---|---|---|---|
| | Protein | Activity | Activity | Purification |
| Step | (mg) | (μg/h) | (μg/h/mg) | (fold) |
| Crude Extract | 343.0 | 22,130 | 64.5 | 1 |
| Ammonium | 275.4 | 21,330 | 77.4 | 1.2 |
| Sulfate 30% | | | | |
| Butyl-sepharose | 33.0 | 13,700 | 415.2 | 6.4 |
| Heparin-agarose | 15.0 | 10,320 | 688.0 | 10.7 |
| 1^{st} UnoQ | 3.6 | 9,690 | 2,691.7 | 41.7 |
| (0-0.3 M NaCl) | | | | |
| 2^{nd} UnoQ | 0.68 | 8,040 | 11,823.5 | 183.3 |
| (0.1-0.25 M NaCl) | | | | |
| 3^{rd} UnoQ | 0.15 | 2,300 | 15,333.3 | 237.7 |
| (0.175-0.3 M NaCl) | | | | |

The N-terminal sequence of the amino acid dehydrogenase is MXSXAAVAFKPGEPLXIV (SEQ ID NO: 14), which is homologous to many different alcohol dehydrogenases. Trypsin digestion of the protein and internal sequences was determined, which indicated homology with known formaldehyde dehydrogenases from Salmonella and Acinetobacter.

Based on this homology, it was determined that the sequences for the N-terminus and one of the internal fragments overlapped. The other internal sequence was presumed to lie approximately 300 amino acids (900 base pairs) downstream. Oligonucleotides for amplification of this gene and subsequent cloning into pBMS2004 by the Polymerase Chain Reaction (PCR) were prepared:

TABLE 7

| Primer | | |
|---|---|---|
| name | Sequence | Description |
| Oligo | GARATHGAYGTIGARGAR | N-terminal region |
| 628 | CCIAA | (EIDVEEPK) of Sporosercina |
| | (SEQ ID NO: 15) | ureae amino acid dehydro- |
| | | genase (sense) |
| Oligo | CCIGTIACIARYTGRAAI | Interal sequence (PFQLVTG) |
| 629 | GG | Sporosarcina ureae amino |
| | (SEQ ID NO: 16) | acid dehydrogenase |
| | | (anti-sense) |

Oligo pair 628+629 was tried with the FailSafe series of PCR buffer (Epicentre Technologies) and Sporosarcina ureae chromosomal DNA as template. The PCR (10 μL final volume) was carried out in a Hybaid PCR Express thermocycler with the following parameters:

TABLE 8

| Stage 1 | Stage 2 | Stage 3 | Stage 4 | Stage 5 | Stage 1 |
|---|---|---|---|---|---|
| 94° 1 min | 94° 30 sec | 94° 30 sec | 94° 30 sec | 72° 5 min | 94° 1 min |
| | 55° 30 sec | 55°-->40° | 40° 30 sec | | |
| | | 30 sec | | | |
| | 72° 30 sec | 72° 30 sec | 72° 30 sec | | |

A fragment of the expected size (˜900 base pairs) was seen upon electrophoresis on a 1.0% TAE agarose gel in FailSafe buffers “B”, “E”, and “F”. The reaction was scaled up 20-fold using buffer F and the entire sample was electrophoresed on a 1.0% agarose gel for 1 hr at 100 v. The amplified fragment was excised from the gel and purified using a Qiagen MinElute™ kit. It was ligated to plasmid pTOPO-TA using 2 μL of the fragment according to the protocol supplied by the vendor (Invitrogen). Two IL of the reaction was used to transform chemically-competent TOP 10 E. coli cells (Invitrogen). SOC medium was immediately added (0.96 ml; SOC=0.5% yeast extract, 2% tryptone, 10 mM NaCl, 2.5 mM KCl, 10 mM MgCl₂, 10 mM MgSO₄, and 20 mM glucose per liter), and the cells were incubated in a shaker for 1 hr at 37° C. and 225 rpm. Colonies containing recombinant plasmids were selected on LB agar plates containing 50 μg/ml kanamycin sulfate (Sigma Chemicals, St. Louis, Mo.). Eighteen Km^{R} colonies were tested for presence of the amino acid dehydrogenase gene by colony using FailSafe buffer “F” and File A6. Following electrophoresis, all colonies except one supported amplification of the 900-bp fragment. Four such colonies were inoculated into TB Km medium and grown at 37° C., 300 rpm, for 20 hr. Plasmid was purified using the Fast Prep kit (Eppendorf). After digestion of about 200 ng of plasmid with EcoRI, all four gave a 3900-bp vector band and a 900-bp insert fragment. DNA sequencing of the insert indicated strong homology to formaldehyde dehydrogenases from other bacteria. The plasmid was named pTOPO-TA-628-629.

A probe specific for the amino acid dehydrogenase was prepared. For labeling, the PCR DIG Probe Synthesis kit (Roche Biochemicals) was used. The reaction mix contained PCR buffer, DIG dNTP mix (dATP, dCTP, dGTP, and dUTP-digoxigenin), 0.4 nM each oligonucleotide, 2.5 U Expand DNA polymerase, and 10 ng of pTOPO-TA-628-629 plasmid DNA. Thermocycling conditions were as follows:

TABLE 9

| | | |
|---|---|---|
| 94° 4 min | 94° C. 30 sec | 72° C. 5 min |
| 1 cycle | 50° C. 30 sec | 1 cycle |
| | 72° C. 1 min | |
| | 30 cycles | |

A sample (5 μL) of the amplified DNA was electrophoresed on a 1.0% agarose gel in TAE buffer for 1 hr at 100 v. A fragment with a higher molecular weight than unlabeled control (due to incorporation of dUTP-digoxigenin) was seen. S. ureae chromosomal DNA was prepared and 3 μg samples digested with a series of restriction endonucleases: Apa I, BamHI, BglII, EcoRI, EcoRV, HindIII, KpnI, NotI, PstI, and SpeI. They were electrophoresed for 18 hr at 20 v and transferred to a nylon filter using the VacuGene™ unit (Amersham Biosciences). The digoxigenin-labeled probe (5 μL) was hybridized to the filter in EasyHyb buffer (Roche Biochemicals) for 18 hr at 42° C., then processed according to the manufacturer's recommended protocol. Stringent wash conditions were 0.5×SSC/0.1% SDS, 65° C. Upon washing and detection, strong hybridization could be seen in all lanes of the filter.

To identify the complete amino acid dehydrogenase gene, a procedure called inverse PCR was used. This involves self-ligation of restriction fragments presumed to contain the complete gene followed by amplification using primers directed away from the gene of interest (thus the term inverse PCR). The PCR fragment is isolated and cloned into a plasmid vector for sequencing. The 5′ and 3′ ends of the insert should contain the ends of the desired gene. A protocol is given below:
1. Based on Southern blot results, select enzyme(s) giving smallest fragment that contains gene of interest and digest 10 μg of chromosomal DNA with 50 U endonuclease in 100 μL final volume for >2 hr
2. Extract with equal volume phenol:chloroform; centrifuge; and retain upper fraction.
3. Precipitate DNA by addition of 0.1 vol. 3 M sodium acetate and 2 vol. EtOH. Centrifuge 5-10 min.
4. Remove liquid, wash pellet with 70% EtOH, and dry in SpeedVac. Resuspend DNA to 100 ng/μL with dH₂O.

5. Set up ligation:

| | | |
|---|---|---|
| Digested DNA | 2.0 | μL (200 ng) |
| 10× FastLink buffer | 3.0 | μL |
| 10× ATP | 3.0 | μL |
| dH₂O | 20.0 | μL |
| Ligase | 2.0 | μL |

Incubate at room temperature, 15 min. Add 45 μL dH₂O+750 μL 1-butanol, centrifuge 10 min. Remove liquid, wash pellet with 70% EtOH, and dry in SpeedVac. Resuspend in 25 μL dH₂O.

6. Set up PCR w/FailSafe

| | |
|---|---|
| Oligo A (100 pmol/μL) | 0.56 μL |
| Oligo B (100 pmol/μL) | 0.56 μL |
| Ligated DNA | 14.0 μL |
| 10× loading dye | 14.0 μL |
| Z-Taq | 0.70 μL |
| dH₂O | 40.2 μL |

Distribute 5 μL to tubes or PCR plate, add 5 μL each FailSafe buffer.
7. Conduct PCR (94° C. 30 sec, 50° C. 30 sec, 72° C. 2 min). Fragment size will be size detected by probe minus expected size of gene not included by primers.
8. Isolate fragment, clone into sequencing vector. Unknown end regions are adjacent to plasmid sequences.

Two restriction endonucleases (EcoRI and HindIII) were chosen for step 1 above based on the size of the hybridizing fragment obtained after Southern blot analysis (about 4300 and 4000 bp, respectively). The primers used for amplification were based on the sequence obtained for the PCR fragment using oligos 628+629:

TABLE 10

| Primer | | |
|---|---|---|
| name | Sequence | Description |
| Oligo | CACCAGCGCCTTCATGAC | Amino acid dehydrogenase |
| 648 | CTAAC | sequencing primer nt |
| | (SEQ ID NO: 17) | 360-337 (anti-sense) |
| Oligo | GCCGCTCTTGAATGCTGC | Amino acid dehydrogenase |
| 649 | C | sequencing primer nt |
| | (SEQ ID NO: 18) | 977-996 (sense) |

In both instances, a fragment of the expected molecular weight was seen upon electrophoresis on a 1.0% TAE agarose gel. The fragment obtained following HindIII digestion/ligation was chosen for further work. It was isolated from the gel and purified using the QIAquick Gel Isolation kit (Qiagen) and ligated to plasmid pTOPO-TA (Invitrogen) using 200 ng of the fragment and 1 μL vector. Two IL of the reaction medium was used to transform chemically-competent TOP 10 E. coli cells (Invitrogen). SOC medium was immediately added (0.96 ml; SOC=0.5% yeast extract, 2% tryptone, 10 mM NaCl, 2.5 mM KCl, 10 mM MgCl₂, 10 mM MgSO₄, and 20 mM glucose per liter), and the cells incubated in a shaker for 1 hr at 37° C. and 225 rpm. Colonies containing recombinant plasmids were selected on LB agar plates containing 50 μg/ml kanamycin sulfate (Sigma Chemicals, St. Louis, Mo.). Thirty-six Km^{R} colonies were tested for presence of the amino acid dehydrogenase gene by colony using FailSafe buffer “E”, and oligos 648+649:

TABLE 11

| | | | |
|---|---|---|---|
| PCR | 94° 4 min | 94° 30 sec | 72° 5 min |
| | 1 cycle | 50° 30 sec | 1 cycle |
| | | 72° 2 min | |
| | | 30 cycles | |

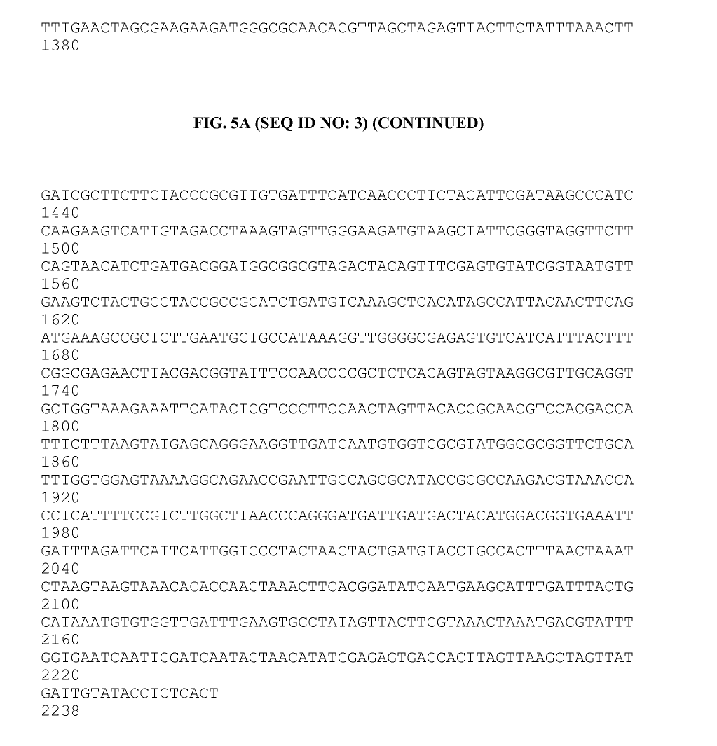

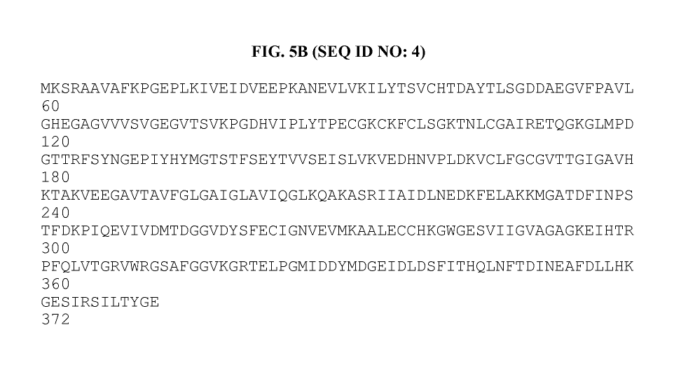

Following electrophoresis, all colonies except one supported amplification of a ˜3000-bp fragment. Four such colonies were inoculated into TB Km medium and grown at 37° C., 300 rpm, for 20 hr. Plasmids were purified using the Fast Prep kit (Eppendorf). After digestion of about 200 ng of plasmid with EcoRI and gel electrophoresis, pairs of plasmids were found to have identical restriction patterns and the non-identical plasmids were probably in the opposite orientation in pTOPO-TA. All four plasmids were submitted for sequencing, which allowed identification of the ends of the amino acid dehydrogenase (called “SUAAD” for Sporosarcina ureae amino acid-dehydrogenase). The complete DNA sequence was obtained and shown in FIG. 5A (SEQ ID NO:2):

Construction of Expression Plasmid pBMS2000-SUAAD:

Primers with appropriate restriction sites for ligation to pBMS2000 and the 5′ or 3′ end of the SUAAD gene were prepared:

TABLE 12

| | |
|---|---|
| Oligo 654 | SwaI/NdeI/N-terminus of |
| GACATATTTAAATCATATGAAAAG | Sporosarcina ureae |
| TAGAGCAGCTGTAGC | Amino acid dehydrogenase |
| (SEQ ID NO: 19) | (sense) |
| Oligo 655 | SwaI/BamHI/C-terminus of |
| CATGATTTAAATGGATCCTCACTC | Sporocarcina ureae |
| TCCATAAGTTAGTATTGATCG | Amino acid dehydrogenase |
| (SEQ ID NO: 20) | (anti-sense); NdeI site |
| | eliminated |

Takara Z-Taq polymerase and included buffer and dNTP mix were used with the above primers, S. ureae chromosomal DNA as template (1 μg), in a 200 μL final volume:

TABLE 13

| | | | |
|---|---|---|---|
| PCR | 94° 4 min | 94° 1 min | 72° 5 min |
| | 1 cycle | 50° 1 min | |
| | | 72° 1 min 30 sec | |
| | | 30 cycles | |

The amplified fragment was purified using a QIAquick Gel Extraction column by first adding 5 vol. of buffer PB (Qiagen) to the reaction mix. Two μg of the purified PCR fragment was digested with 10 U each of restriction endonuclease NdeI and BamHI in a final volume of 40 μL for 2 hr at 37° C. and electrophoresed on a 1.0% agarose gel in TAE buffer for 1 hr at 100 v. The digested fragment was again purified on a QIAquick column as described above, then ligated to NdeI+BamHI-digested pBMS2004 in a 15 μL reaction volume using the FastLink kit from Epicentre. Two eliminate vector-only plasmids, 0.5 μL KpnI (5 U) was then added and the reaction incubated at 37° C. for 30 min. DNA was precipitated by addition of 22.5 μL dH₂O and 375 μL 1-butanol, then centrifuged 5 min at 13,500×g. Liquid was removed and the pellet washed with 200 μL 70% EtOH. Ethanol was removed and the pellet dried for 2 min in a SpeedVac. DNA was resuspended in 4 μL dH₂O and used to transform electrocompetent DH10B cells colonies, which were selected on LB+Km agar medium. PCR of selected transformants using oligos 654+655 revealed 3/18 colonies tested possessed the correct insert. Two of these colonies were grown in LB Km liquid medium at 37° C., 250 rpm, for 20 hr, and plasmid DNA isolated. Restriction digestion of 0.2 μg of plasmid DNA with 5 U each NdeI and BamHI as well as EcoRV gave the expected fragments. One plasmid was chosen for further study and named pBMS2004 SUAAD.

Heterologous Expression of SUAAD:

pBMS2000-SUAAD was transformed into chemically-competent E. coli BL21 cells (Stratagene) according to the manufacturer's directions and streaked for single colonies by growth on LB Km agar medium (37° C., 22 hr). A single colony was inoculated into MT5-M2 medium+Km and grown at 30° C. for 20-22 hr, 250 rpm. This culture was used for expression work. SDS-polyacrylamide gel electrophoresis showed overexpression of a protein of the expected molecular weight of 40,000 Daltons.

E. coli BL21 cells (pBMS2000-SUAAD) were grown in MT5-M2 medium containing 2.0% Hy-Pea (Quest International), 1.85% Tastone-154 (Sensient Bionutrients), 0.6% Na₂HPO₄, 0.125% (NH₄)₂SO₄, and 4.0% glycerol, adjusted to pH 7.2 pre-sterilization. Using a 50 mg/mL filter-sterilized aqueous stock solution, kanamycin sulfate was aseptically added to a level of 50 μg/mL after autoclaving. For shake flask expression work, cells were initially grown in MT5-M2 for 20-24 hr at 30° C. and 250 rpm. The optical density at 600 nm (OD₆₀₀) was recorded and fresh medium was inoculated with the culture to a starting OD₆₀₀ of 0.30. The flask was incubated as described above until the OD₆₀₀ reached ˜0.8-1.0. Isopropyl-thio-β-D-galactoside (IPTG) was added from a 1 M filter-sterilized stock in de-ionized H₂O to the desired final concentration (50 μM or 1 mM) and the culture was allowed to grow for varying lengths of time before harvesting by centrifugation.

Racemic Conversion:

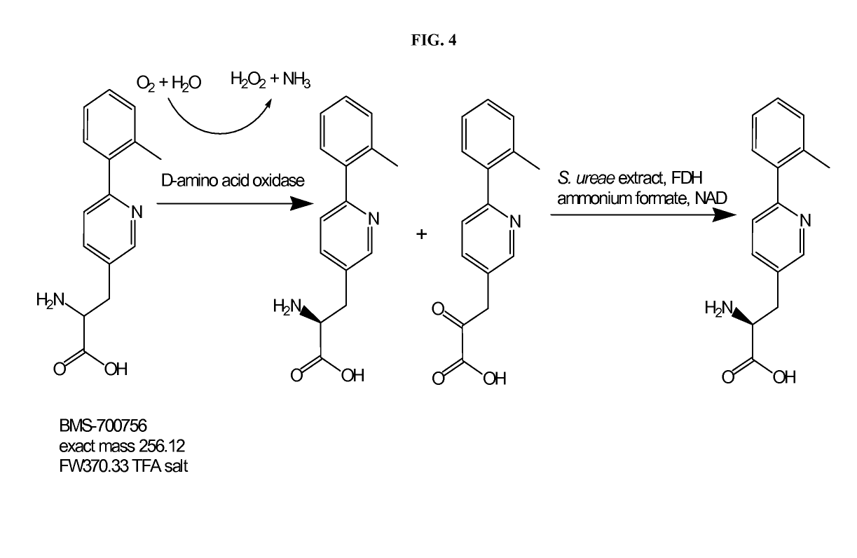

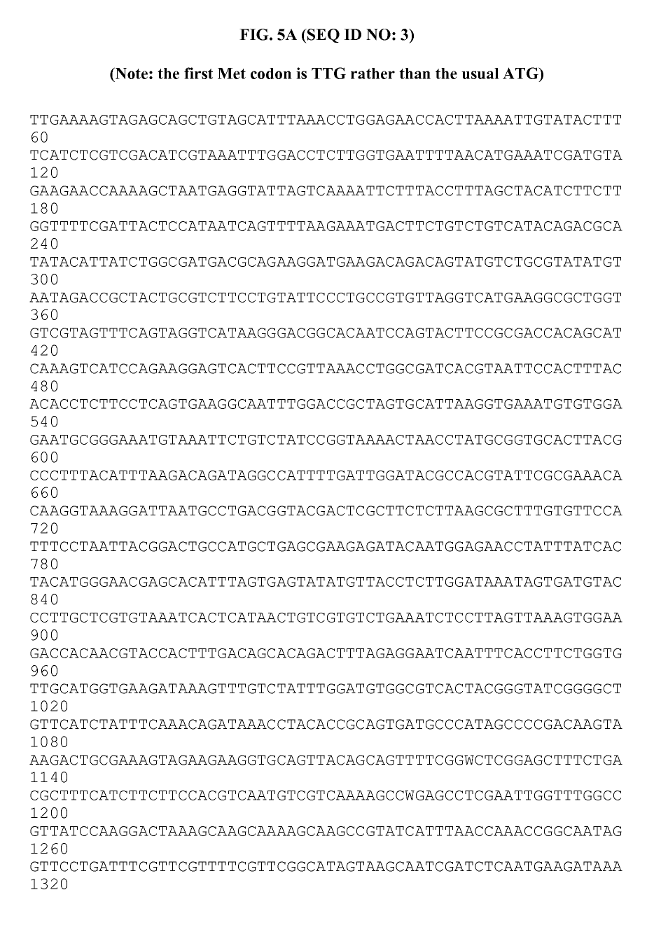

Racemic amino acid was first converted to (S)-amino acid and the corresponding keto acid by treatment with D-amino acid oxidase from Trigonopsis variabilis. Subsequently, the keto acid is converted to the corresponding (S)-amino acid by addition of ammonium formate, NAD, dithiothreitol and an extract of Sporosarcina ureae SC16048 (See FIG. 4). Using the oxidase followed by the S. ureae extract, 200 mg racemic amino acid (BMS-700756-01) was converted to 171 mg (86% yield) of the (S)-enantiomer. Thus in overall reaction, the racemate is converted completely to (S)-amino acid in e.e. >99.5% Similar results were obtained when recombinant Escherichia coli (pBMS2000-SUAAD) cell extracts expressing amino acid dehydrogenase from Sporosarcina ureae SC16048 was used.

I. Organisms

Two recombinant strains of E. coli were used for the following process descriptions. Strain SC16541 [E. coli BL21(pBMS2000-AAOA)] was used for the production of adenosylmethionine-8-amino-7-oxononanoate aminotransferase (AAOA). AAOA from Burkholderia species was cloned and expressed in Escherichia coli, as described below.

Strain SC16544 [E. coli BL21(pBMS1000 DAAO)] was used for the production of recombinant D-amino acid oxidase (DAAO). DAAO from Trigonopsis variabilis was cloned and expressed in Escherichia coli, as described (UniProtKB/Swiss-Prot entry Q99042).

The expression vectors used for both constructs include promoters that can be induced by IPTG.

II. Fermentation of SC16541 (AAOA aminotransferase) Inoculum:

Two frozen vials of SC16541 were thawed and the entire contents (1.5 ml) were transferred to each of two 500 ml flasks containing 100 ml of MT5 medium (see table below). The F1 stage flasks were incubated at 30° C. for 24 hours and 250 rpm. From each F 1 flask, 5 ml were transferred to two 4-liter flasks containing 1 liter of the same MT5 medium (total: 4 flasks). The four F2 flasks were then incubated at 30° C. and 230 rpm for an additional 18-20 hours. As necessary, and as utilized in one of the examples below (Tank batch #5B03433), an additional 4-liter F3 stage may be used, with no adverse effect upon cell yield or activity. The F2 and F3 stages for this particular batch were incubated for 24 and 18 hours, respectively.

The four F2 (or F3) flasks were pooled and the OD₆₀₀ was measured. For the 3 runs described here, the average inoculum OD₆₀₀ was 5.5 U/ml. The 4 liters of pooled inoculum were then transferred to a 380-liter tank containing a working volume of 250 liters of MT5-M2 medium (see table below).

250-Liter Tank Process Conditions:

The recommended fermentation process conditions for a tank containing 250 liters of MT5-M2 medium are as follows:

| | |
|---|---|
| Temperature: | 30° C. |
| pH control: | none |
| Agitation: | 300 rpm |
| Aeration: | 250 lpm |
| Dissolved oxygen: | no control |
| Back pressure: | 10 psi |
| Antifoam: | UCON ™ Lubricant LB-625 |
| Induction: | IPTG added at a level of 50 μM (about log 5.5 |
| | hours) |
| Harvest: | about 24-25 hours |

Following inoculation, optical density was measured hourly to determine induction time. At an OD₆₀₀ of about 0.8 to 1.0 U/ml, the IPTG solution was added to yield a final concentration of 50 μM. This addition time corresponded to a CO₂ off-gas value of about 0.11-0.15.

Fermentation Final Cell Density and Activity:

Cell density range: OD₆₀₀: 44-48
Cell recovery: 11-14 kg

Aminotransferase activity: 0.7-0.8 U/g cells

TABLE 14
MT5 flask medium (SC16541)

| | | Concentration |
|---|---|---|
| Component | Source | (%) |
| Yeastamin 95 | Tate & Lyle (Staley) | 2.0 |
| Glycerol | Mallinkrodt 5092 | 4.0 |
| Sodium phosphate, dibasic | Mallinkrodt | 0.6 |
| Potassium phosphate, monobasic | J T Baker | 0.3 |
| Ammonium sulfate | J T Baker | 0.125 |
| Magnesium sulfate, heptahydrate | Sigma | 0.0246 |
| Kanamycin | Sigma | 0.005 |

TABLE 15
MT5-M2 tank medium (SC16541)

| | | Concentration |
|---|---|---|
| Component | Source | (%) |
| Hy-Pea 7404 | Quest International | 2.0 |
| Tastone 154 | Sensient Bionutrients | 1.85 |
| Glycerol | Dow OPTIM 99.7% USP | 4.0 |
| Sodium phosphate, dibasic | J T Baker | 0.6 |
| Ammonium sulfate | J T Baker | 0.125 |
| UCON antifoam LB-625 | Dow Chemical | 0.04 |
| Kanamycin | Sigma | 0.005 |

TABLE 16
IPTG

| Component | Source | Concentration |
|---|---|---|
| IPTG, dioxane free | Calbiochem | 50 μM |

For IPTG, 2.98 g was dissolved in 500 ml de-ionized water, filter-sterilized, and added to a transfer bottle. The IPTG solution was transferred to the tank medium when growth reached an OD₆₀₀ of about 0.8-1.0

Harvest:

At the end of the run (about 24-25 hours post-inoculation), the whole broth was harvested by centrifugation. At this time, CO₂ off-gas had already peaked and was starting a slow decline. During centrifugation, the cells were washed with 50 mM pH 7.0 potassium phosphate buffer. The cell paste was then stored at −70° C. until ready for the next processing step.

III. Fermentation of SC16544 (D-Amino Acid Oxidase) Inoculum:

Two frozen vials of SC16544 were thawed and the entire contents (1.5 ml) were transferred to each of two 500 ml flasks containing 100 ml of MT5 medium (see table below). The F1 stage flasks were incubated at 28° C. for 24-25 hours and 250 rpm. From each F1 flask, 5 ml were transferred to two 4-liter flasks containing 1 liter of the same MT5 medium (total: 4 flasks). The four F2 flasks were incubated at 28° C. and 230 rpm for an additional 22-24 hours. Note: This strain grows more slowly than SC16541 under these conditions, and an extended F2 incubation period is required. Also note other significant differences in process conditions versus SC16541.

The four F2 flasks were pooled and the OD₆₀₀ was measured. For the 2 runs described here, the average inoculum OD₆₀₀ was 3.6 U/ml. The 4 liters of pooled inoculum were then transferred to a 380-liter tank containing a working volume of 250 liters of MT5-M2 medium (see table below).

250-Liter Tank Process Conditions:

The recommended fermentation process conditions for a tank containing 250 liters of MT5-M2 medium are as follows:

| | |
|---|---|
| Temperature: | 28° C. |
| pH control: | at or above pH 7.2 with ammonium hydroxide |
| Agitation: | 300 rpm |
| Aeration: | 250 lpm |
| Dissolved oxygen: | no control |
| Back pressure: | 10 psi |
| Antifoam: | UCON ™ Lubricant LB-625 |
| Induction: | IPTG added at a level of 150 μM (about log 5.5-6.0 |
| | hours) |
| Harvest: | about 20-21 hours |

Following inoculation, optical density was measured hourly to determine induction time. At an OD₆₀₀ of about 1-2 U/ml, the IPTG solution was added to yield a final concentration of 150 μM. This addition time corresponded to a CO₂ off-gas value of about 0.10-0.23.

Fermentation Final Cell Density and Activity:

Cell density range: OD₆₀₀: 14-23

Cell recovery: 6.8-7.2 kg

D-amino acid oxidase activity: about 33-39 U/g cells

TABLE 17
MT5 flask medium (SC16544)

| | | Concentration |
|---|---|---|
| Component | Source | (%) |
| Yeastamin 95 | Tate & Lyle (Staley) | 2.0 |
| Glycerol | Mallinkrodt 5092 | 4.0 |
| Sodium phosphate, dibasic | Mallinkrodt | 0.6 |
| Potassium phosphate, monobasic | J T Baker | 0.3 |
| Ammonium sulfate | J T Baker | 0.125 |
| Magnesium sulfate, heptahydrate | Sigma | 0.0246 |
| Kanamycin | Sigma | 0.003 |

TABLE 18
MT5-M2 tank medium (SC16544)

| | | Concentration |
|---|---|---|
| Component | Source | (%) |
| Hy-Pea 7404 | Quest International | 2.0 |
| Tastone 154 | Sensient Bionutrients | 1.85 |
| Glycerol | Dow OPTIM 99.7% USP | 4.0 |
| Sodium phosphate, dibasic | J T Baker | 0.6 |
| Ammonium sulfate | J T Baker | 0.125 |
| UCON antifoam LB-625 | Dow Chemical | 0.04 |
| Kanamycin | Sigma | 0.003 |

TABLE 19
IPTG (SC16544)

| Component | Source | Concentration |
|---|---|---|
| IPTG, dioxane free | Calbiochem | 150 μM |

For IPTG, 8.93 g was dissolved in 500 ml de-ionized water, filter-sterilized, and added to a transfer bottle. The IPTG solution was added to the tank medium when growth reached an OD₆₀₀ of about 1-2.

Harvest:

At the end of the run (about 20-21 hours post-inoculation), the whole broth was harvested by centrifugation. At this time, CO₂ off-gas had already peaked and was starting a slow decline. During centrifugation, the cells were washed with 10 mM pH 7.0 potassium phosphate buffer. The cell paste was then stored at −70° C. until ready for the next processing step.

TABLE 20
SUMMARY OF FERMENTATION RESULTS

| | | | | | Cell | |
|---|---|---|---|---|---|---|
| | | Tank | | Cell | Yield | Activity |
| Strain | Enzyme | Batch # | Cell PO # | Batch # | (kg) | (U/g cells) |
| SC16541 | Aminotransferase | 5A09845 | 101051574 | 5A09850 | 11.205 | 0.725 |
| SC16541 | Aminotransferase | 5B03433 | 101051484 | 5A10019 | 11.250 | 0.775 |
| SC16544 | D-aa oxidase | 5B04053 | 101055596 | 5B04075 | 7.134 | 33.40 |
| SC16541 | Aminotransferase | 5B06132 | 101058784 | 5B06142 | 13.312 | 0.784 |
| SC16544 | D-aa oxidase | 5B07585 | 101060214 | 5B07591 | 6.842 | 38.75 |

Preparation of Cell Free Extracts:

For preparation of cell-free extracts, 200 g (wet weight) of SC16541 cells containing AAOA S-aminotransferase and 40 g (wet weight) of SC16544 containing R-amino acid oxidase were suspended in 760 ml of 100 mM pH 7.0 potassium phosphate buffer containing 5 μM Pyridoxal 5′-phosphate (PLP) a 24% cell suspension, then disintegrated with a Microfluidizer at 10,000 psi for 3 passes. To the mixture, 5 g of 50% polyethyleneimine (PEI) was added to a final concentration of 0.2% and mixed well. The PEI treated mixture was centrifuged at 15,000×g (GSA rotor, Sorvall) for 2 min at 4° C. to remove the cell debris. The supernatant was collected and stored at 4° C. for subsequent enzymatic reactions.

Bioconversion at 1 Liter Scale:

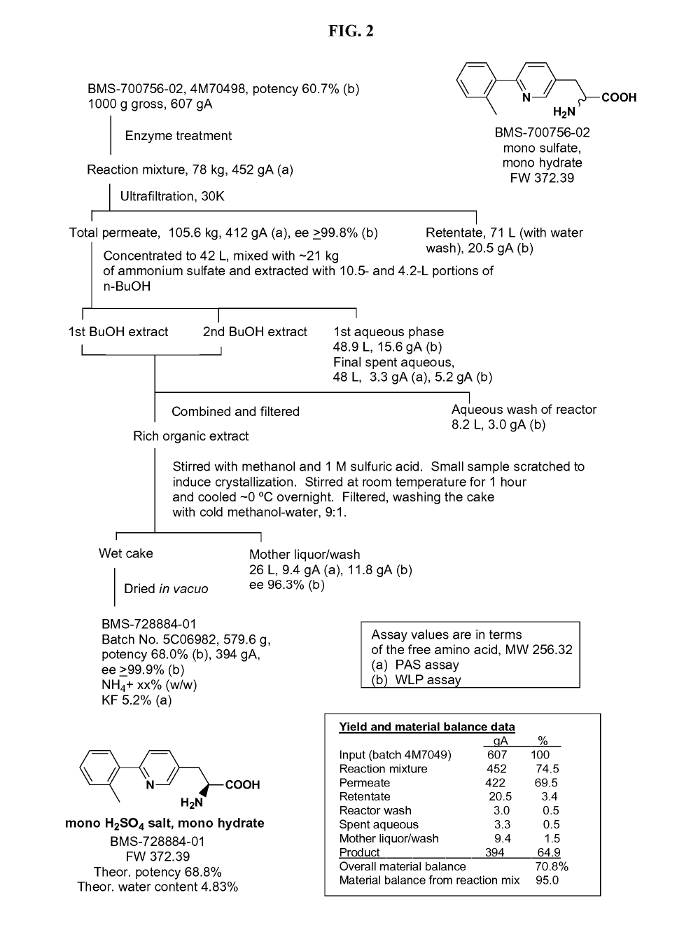

Bioconversion of racemic amino acid BMS-700756-02 (monosulfate monohydrate) to 2-(S)-amino acid: [BMS-728884] 2-S-amino-3-[3-{6-(2-methylphenyl)}pyridyl]-propionic acid was conducted at the 1 liter scale with 15 g of substrate input. First, 15 g of BMS-700756-02, 25 g of sodium aspartate and 5 g of ascorbic acid were dissolved in 50 ml of propylene glycol and 430 ml of water. The solution was adjusted to pH 7.5 with 10 N NaOH while stirring, and poured in to a 2-liter reaction vessel. Next, 2 ml of catalase (392 KU), 500 ml of cell extract containing 165 units of R-amino acid oxidase, and 72.5 units of AAOA transferase were added to the vessel. Finally, 2 ml of antifoam SAG-5693 was added to the reaction mixture. The reactor was controlled by a Braun Biostat B with the following settings: temperature 30° C., agitation 300 rpm, pH 7.5 and aeration at 1 vvm. Samples were taken at every hour to analyze optical purity and conversion yield. The aeration was stopped at 6 hours when the oxidation was complete by judging ee>99%. The reaction continued for an additional 16 hours without aeration, and the reaction mixture was harvested at 22 hours. The reaction was completed at 22 hours with 84.9% conversion yield and ee>99.5%. The reaction mixture was ultrafiltered extracted with butanol and ammonium sulfate, and finally crystallized (See FIG. 2). The overall yield was 72% after product isolation.

HPLC Methods:

Methods for estimating bioconversion yield and ee of product.

1. Achiral method to quantify free amino acid (BMS-700756)
Column: YMC Pak ODS A 15×0.46 cm
Mobile phase: A. 0.05% TFA in water; B. 0.05% TFA in acetonitrile
Gradient: 0-12 min, 0-80%
Flow rate: 1 ml/min
Column temperature: 40° C.
Detection: 282 nm
Injection volume: 10 μl
Retention times: BMS-700756: 5.4 min; BMS-728884: 5.4 min; Keto acid: 6.6 min.

2. Chiral method (a) to determine ee
Column: Chirobiotic T 25×0.46 cm
Mobile phase: A. water:methanol 90: 10:05% acetic acid; B. methanol:water 90:10: 0.05% acetic acid
Gradient: 0-3 min, 10% B; 3-15 min, 10-100% B; 15-20 min, 100% B
Flow rate: 1.2 ml/min
Column temperature: 25° C.
Detection: 215 nm
Injection volume: 5 μl
Retention times: S-enantiomer (BMS-728884): 14.1 min; R-enantiomer of BMS-700756: 14.9 min

3. Determination of ee after derivatization to diastereomers
Reaction mixture (10 μl) containing ˜5-10 mg/ml BMS-700756 was reacted with 8 μl of 1 M NaHCO3 and 40 μl of 1% (w/v) Marfey's reagent (1-fluoro-2,4-dinitrophenyl-5-L-alanine amide)) in acetone by incubating at 40° C. for 1 hour. The reaction was quenched by adding 8 μl of 1 N HCl and 934 μl of acetonitrile-water (1:1), an the resulting solution was analyzed by HPLC. The HPLC method to determine ee after derivatization was as follows:.
Column: YMC Pak ODS A 15×0.6 cm; Mobile phase: A. 0.05% TFA in water; B. 0.05% TFA in acetonitrile; Isocratic elution: 25% B, 20 min; Flow rate: 1.5 ml/min; Column temperature: 40° C.;
Detection: 340 nm; Injection volume: 10 μl; Retention times: S-enantiomer 10.5 min; R-enantiomer 15.3 min.

The bioconversion process was scaled up in the pilot plant to convert 1 kg racemic amino acid [BMS-700756-02] to (S)-amino acid: [BMS-728884]2-S-amino-3-[3-{6-(2-methylphenyl)}pyridyl]-propionic acid.

1. Preparation of Cell Extract

Frozen SC16541 cells (12 kg) and SC16544 cells (2.4 kg) were thawed and suspended in 46 liters of 0.1 M phosphate buffer (pH 8.0) containing 5 μM PLP with agitation at 200 rpm. The 24% cell suspension was passed twice through a 610EG microfluidizer to obtain 85 liters of mixture (a larger than planned volume was due to the dilution by water, and temperature of the reaction vessel exceeded 25° C. for more than 3 5 hours). To the mixture, 340 ml of 50% PEI_was added and mixed for 30 minutes, followed by centrifugation with a Sharples centrifuge at 0.4 gal/min to clarify the extracts.

2. Bioconversion

To the 100 L vessel containing 28 liters of water, 1.67 kg sodium aspartate and 0.33 kg ascorbic acid were charged. Next 1 kg BMS-700756-02 with 3.33 kg propylene glycol was then charged while agitating at 100 rpm. The solution was adjusted to pH 7.0 by adding 25% NaOH. Next, 0.13 kg catalase, 167 mg PLP, 40 L clarified extract and 133 g SAG-5693 were added to the vessel sequentially. The reaction was run at 30° C., 250 rpm, pH 7.5 and 80 LPM. Samples were taken every 2 hours to assay enantiomeric excess of product and conversion yield by chiral and achiral HPLC methods, respectively.

The bioconversion was completed after 25 hours at 30° C., 250 rpm, pH 7.5 and aeration at 1 vvm for 4 hours to give 74.5% yield and ee>99.9%. The oxidation in the 100-liter reactor was completed within 2 hours and ee reached 99.9%, which may be due to more efficient agitation and aeration in the larger vessel. The reaction mixture was ultrafiltered and worked up to obtain 579.6 g BMS-728884-01 with an overall yield of 64.9%. (See FIG. 2).

Conversion with Enzymes from BioCatalytics:

Reactions were carried out in 15 ml tubes incubated at 30° C. with shaking at 300 rpm. Reactions contained immobilized D-amino acid oxidase (20 mg, 0.6 units), BMS-700756 (15 mg, 61.2% potency, 35.8 μmoles), catalase (392 units), 100 mM L-glutamate, 0.1 mM pyridoxal phosphate and either broad range transaminases AT-101 or transaminase AT-106 (1.5 mg) in 1 ml 0.1 M potassium phosphate buffer, pH 7.5. Yields determined by HPLC were 80.2% and 69.8% from incubations with AT-101 and AT-106, respectively. Ee's, determined with the Marfey reagent as described above, were 100% in both samples. All enzymes were obtained from BioCatalytics, Inc.

Conversion with R-Amino Acid Oxidase and Amino Acid Dehydrogenase

For preparation of Sporosarcina ureae cells containing amino acid dehydrogenase, the growth/fermentation medium was as follows: 1.0% L-phenylalanine, 1.0% peptone, 0.5% yeast extract, 0.2% K₂HPO₄, 0.1% NaCl, 0.02% MgSO₄ adjusted to pH 7 with K₂HPO₄ or KH₂PO₄. Broth (0.5 ml) from frozen vials containing Sporosarcina ureae SC16048 was used to inoculate 100 ml medium in each of four 500-mL flasks. After incubation at 28° C., 200 rpm for two days 200 ml of the culture was used to inoculate 15 L medium in each of two fermentors. Fermentation was carried out at 1 vvm airflow, 500 rpm agitation and 28° C. No pH control was used during the fermentation. After 48 h, cells were collected with a Sharples centrifuge, washed with 10 mM potassium phosphate buffer pH 7, then stored at −70° C. until used. 412 g cell paste was recovered from the two tanks.

For preparation of extracts from Sporosarcina ureae SC16048, cells (20 g, stored frozen at −70° C.) were suspended in 50 mM ammonium formate, pH 8 containing 1 mM dithiothreitol, to a volume of 100 ml using an Ultraturrax homogenizer, then disrupted by sonication for 3 minutes. The sonicate was centrifuged at 27491×g for 20 min., and the supernatant used as a source of amino acid dehydrogenase.

BMS-700756 (2.000 g, 7.809 mmoles) was dissolved in 95 ml water. 5 ml 1 M potassium phosphate buffer pH 7 was added and the solution was adjusted from pH 8.67 to pH 8 with a few drops of 99% formic acid. Immobilized D-amino acid oxidase (Trigonopsis variabilis enzyme expressed in E. coli and immobilized on celite, 4 g, 280 units) and catalase (0.2 ml, 7840 units from BioCatalytics) were added and the mixture was shaken at 28° C., 250 rpm in a 250 ml flask. After 5.5 h the ee was 89.2% by the Marfey procedure.

After 7.5 h, ammonium formate (2.524 g, 40.02 mmoles), dithiothreitol (30.8 mg, 0.2 mmole), NAD ((66.34 mg, 0.1 mmole), formate dehydrogenase (20 mg, 10 units of yeast enzyme from Boehringer) and extract from Sporosarcina ureae SC16048 were added and the pH was adjusted to 8.05 with 1N NaOH. The flask was incubated at 28° C., 40 rpm for 19 h at which time the broad HPLC peak corresponding to the keto acid was no longer present. The flask was placed in a boiling water bath for 5 minutes to coagulate proteins, cooled to room temperature, then centrifuged at 27491×g for 20 min. The pellet washed with 20 ml water and centrifuged again. The combined supernatants contained 1.355 g BMS-728884 (by HPLC based on the input material as a standard), ee near 100% in 176 ml.

The reaction mixture (176 mL), containing 1.255 g of BMS-728884 (in terms of the neat amino acid), was adjusted from pH 7.87 to 6.95 with 10 M H₂SO₄ (0.22 g) and stirred with 35.2 g of Na₂SO₄ until the salt dissolved. The mixture was extracted with 176- and 44-mL portions of n-BuOH, back-washing each extract with a single 44-mL portion of aqueous Na₂SO₄ (0.2 g per mL of water). Rag layers and upper-phase emulsions were centrifuged to obtain good recovery of upper phase.

The combined rich butanol phase (assay: 1.131 g, 4.41 mmoles) was extracted with one 48.5- and two 10-mL portions of 0.1 M H₂SO₄. Assay of the organic phase indicated that 40 mg of BMS-728884 remained.

The combined aqueous phase was concentrated to 11.7 g (crystallization was abundant by 25 g), mixed with 12.1 mL of EtOH, sonicated to give a smooth slurry and left at room temperature for 15 hours. The product was filtered out, washed with 10 mL of EtOH-water, 1:1, and dried in vacuo to constant weight (3 hours) at room temperature, to give 1.7002 g of BMS-728884-01, as a nearly colorless crystalline solid.

TGA showed loss of about 1 mole of water at 110˜155° C. with a corresponding melt endotherm at 150° C. in the DSC. Elemental analysis indicated substantial contamination (9.8%) with Na₂SO₄. The ¹H NMR spectrum, indicated negligible contamination with organic impurities. Based on the elemental analysis for carbon and the lack of other organic impurities by NMR spectroscopy, the potency relative to the neat amino acid is 62.1%.

The ee was ≧99.9% by Marfey assay. Assay of the mother liquor, which contained 60.9 mg of BMS-728884, gave an ee of 94.2%, from which an ee of 99.72% was calculated for the crude product from the enzymatic resolution.

Alternative preparation of extract from Sporosarcina ureae SC16048. Cells (stored frozen at −70° C.) were suspended at a concentration of 20% w/v in 50 mM potassium phosphate buffer, pH 7, containing 1 mM dithiothreitol to a volume 5 ml using an Ultraturrax homogenizer, then disrupted by sonication for 3 minutes. The sonicate was centrifuged at 43152×g for 10 min, and the supernatant was used as a source of amino acid dehydrogenase.

BMS-700756 (100 mg, 0.39 mmoles) was dissolved in 10 ml 50 mM potassium phosphate buffer pH 7 and the solution was adjusted to pH 8 with 10 N NaOH and 1 N HCl. Immobilized D-amino acid oxidase (Trigonopsis variabilis enzyme expressed in E. coli and immobilized on celite, 200 mg, 14 units) and catalase (0.01 ml, 392 units from Biocatalytics) were added and the mixture was shaken at 28° C., 250 rpm in a 50 ml tube. After 2.5 h the ee was 100% by the Marfey procedure.

After 2 h, 1 ml 2 M NH₄OH (2 mmoles, adjusted to pH 8.75 with HCl), NAD (4.0 mg, 6 μmoles), 0.15 ml of 100 mM dithiothreitol, extract from Sporosarcina ureae SC16048 (3 ml containing 35 u phenylalanine dehydrogenase and possibly another amino acid dehydrogenase more active with this substrate) and glucose dehydrogenase (1 mg, 72 units from Amano) were added and the pH was adjusted to 8.75 with 2.8% NH₄OH. The tube was incubated at 30° C. for 20 h at which time the broad HPLC peak corresponding to the keto acid was no longer present. The flask was placed in a boiling water bath for 5 minutes to coagulate proteins, cooled to room temperature, then centrifuged at 43152×g for 10 min. The pellet washed with 2 ml water and centrifuged again. The combined supernatants contained 72.1 mg BMS-728884 (72.1% yield by HPLC based on the input material as a standard), ee near 100% in 17.8 ml.

Dynamic Resolution of BMS-700756:

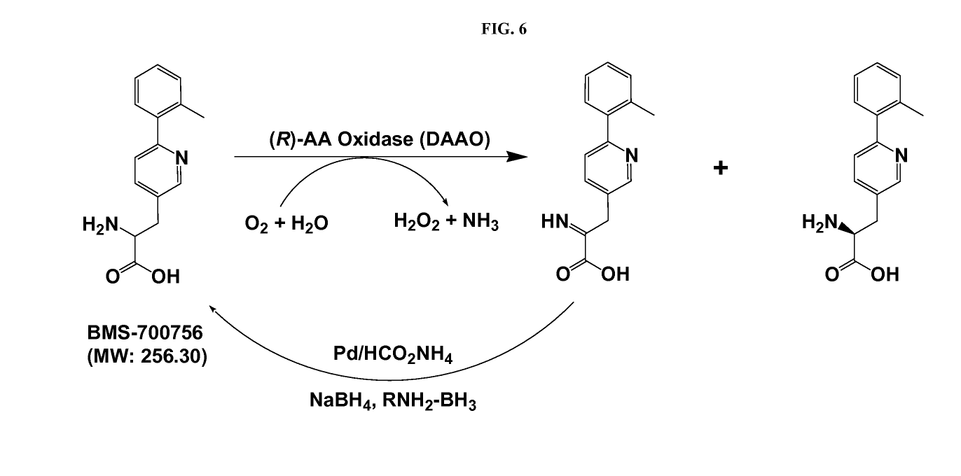

The chemoenzymatic dynamic resolution of BMS-700756 was examined, using (R)-selective oxidation with Celite-immobilized D-Amino Acid Oxidase (DAAO) in combination with chemical imine reduction with borane-ammonia (See FIG. 6). A suspension of BMS-700756 (1.0 mL, about 36 mM, 15 mg/mL of sulphate salt in water, adjusted to pH 5.0, 5.5, 6.0, 6.5, 7.0, 7.5 and 8.0 with 2 M ammonia, and containing 0.2 M sodium phosphate of the appropriate pH together with 0.1 M ammonium formate) was added to a mixture of immobilized DAAO (50 mg, immobilized onto celite with poly(ethylene imine) and glutaraldehyde crosslinking) and borane-ammonia (11 or 22 mg, 10 or 20 mole equivalents) in a 6 mL vial, the vial sealed with breathable sealing film, and the suspension stirred at 400 rpm, RT, 20 h. As seen from Table 7, significant levels of dynamic resolution were observed with reactions carried out at pH>5.5. The yield of the desired (s)-amino acid peaked at pH 6.0-6.5, with E.e. values reaching 100% at pHs above 5.5 (20 h incubation).

TABLE 21
Dynamic Resolution of BMS-700756

| | Substrate | | | Biocatalyst | | Reductant | | | Buffer | | Additive | | | | |
|---|---|---|---|---|---|---|---|---|---|---|---|---|---|---|---|
| | Pot | Conc | | | Load | | Conc | Eqs | | Conc | | Conc | Yield (%) | | E.e. |
| # | (%) | (mg/mL) | (mM) | Type | (g/L) | Type | (mM) | (mol) | pH | (M) | Type | (M) | (S) | (S) + (R) | (%) |
| 1 | 62 | 15 | 36 | DAAO | 50 | NH₃—BH₃ | 180 | 10 | 5.0 | 0.20 | HCO₂NH₄ | 0.10 | 53 | 70 | 51 |
| 2 | 62 | 15 | 36 | DAAO | 50 | NH₃—BH₃ | 180 | 10 | 5.5 | 0.20 | HCO₂NH₄ | 0.10 | 56 | 68 | 82 |
| 3 | 62 | 15 | 36 | DAAO | 50 | NH₃—BH₃ | 180 | 10 | 6.0 | 0.20 | HCO₂NH₄ | 0.10 | 78 | 78 | 100 |
| 4 | 62 | 15 | 36 | DAAO | 50 | NH₃—BH₃ | 180 | 10 | 6.5 | 0.20 | HCO₂NH₄ | 0.10 | 79 | 79 | 100 |
| 5 | 62 | 15 | 36 | DAAO | 50 | NH₃—BH₃ | 180 | 10 | 7.0 | 0.20 | HCO₂NH₄ | 0.10 | 76 | 76 | 100 |
| 6 | 62 | 15 | 36 | DAAO | 50 | NH₃—BH₃ | 180 | 10 | 7.5 | 0.20 | HCO₂NH₄ | 0.10 | 73 | 73 | 100 |
| 7 | 62 | 15 | 36 | DAAO | 50 | NH₃—BH₃ | 180 | 10 | 8.0 | 0.20 | HCO₂NH₄ | 0.10 | 67 | 67 | 100 |
| 8 | 62 | 15 | 36 | DAAO | 50 | NH₃—BH₃ | 360 | 20 | 5.0 | 0.20 | HCO₂NH₄ | 0.10 | 55 | 68 | 81 |
| 9 | 62 | 15 | 36 | DAAO | 50 | NH₃—BH₃ | 360 | 20 | 5.5 | 0.20 | HCO₂NH₄ | 0.10 | 57 | 66 | 86 |
| 10 | 62 | 15 | 36 | DAAO | 50 | NH₃—BH₃ | 360 | 20 | 6.0 | 0.20 | HCO₂NH₄ | 0.10 | 80 | 80 | 100 |
| 11 | 62 | 15 | 36 | DAAO | 50 | NH₃—BH₃ | 360 | 20 | 6.5 | 0.20 | HCO₂NH₄ | 0.10 | 78 | 78 | 100 |
| 12 | 62 | 15 | 36 | DAAO | 50 | NH₃—BH₃ | 360 | 20 | 7.0 | 0.20 | HCO₂NH₄ | 0.10 | 78 | 78 | 100 |
| 13 | 62 | 15 | 36 | DAAO | 50 | NH₃—BH₃ | 360 | 20 | 7.5 | 0.20 | HCO₂NH₄ | 0.10 | 75 | 75 | 100 |
| 14 | 62 | 15 | 36 | DAAO | 50 | NH₃—BH₃ | 360 | 20 | 8.0 | 0.20 | HCO₂NH₄ | 0.10 | 74 | 74 | 100 |

Notes:
Yields and E.e.s were determined by chiral HPLC.
Abbreviations: Pot, Potency; DAAO, D-Amino Acid Oxidase

We claim:

1. Isolated 2-S-amino-3-[3-{6-(2-methylphenyl)}pyridyl]-propionic acid, or a salt thereof.

2. Use of the isolated (S)-enantiomer of 2-(S)-Amino-3-[3-{6-(2-methylphenyl)}pyridyl]propionic acid of claim 1, or a salt thereof, for the preparation of a GLP-1 mimic.

3. Isolated 2-oxo-3-[3-{6-(2-methylphenyl)}pyridyl]-propionic acid, or a salt thereof.

4. A substantially purified polypeptide, comprising an amino acid sequence according to SEQ ID NO:2 or SEQ ID NO:4, or functional equivalents thereof.

5. The subsequently purified polypeptide of claim 4 wherein the polypeptide comprises the amino acid sequence of SEQ ID NO:2.

6. The subsequently purified polypeptide of claim 4 wherein the polypeptide comprises the amino acid sequence of SEQ ID NO:4.

7. An isolated nucleic acid comprising a nucleic acid that encodes the polypeptide of claim 4.

8. The isolated nucleic acid of claim 7, wherein the nucleic acid comprises the sequence of SEQ ID NO: 1 or SEQ ID NO:3.

9. The isolated nucleic acid of claim 8, wherein the nucleic acid comprises the sequence of SEQ ID NO:1.

10. The isolated nucleic acid of claim 8, wherein the nucleic acid comprises the sequence of SEQ ID NO:3.

11. A recombinant expression vector comprising the isolated nucleic acid of any one of claims 7-10.

12. A recombinant host cell comprising the recombinant expression vector of claim 11.

13. A cell extract derived from the recombinant host cells of claim 12.

14. A method for converting a racemic amino acid to an (S) amino acid, comprising (a) reacting a racemic amino acid with an amount of an amino acid oxidase of sufficient to produce a mixture of (S) amino acid and keto acid; and (b) reacting the mixture with (i) an amount of an aminotransferase of SEQ ID NO:2 in the presence of an amino acid, or (ii) an amino acid dehydrogenase of SEQ ID NO:4 in the presence of an amino donor group and a cofactor selected from the group consisting of NADH; NAD+formate+formate dehydrogenase; and NAD+glucose+glucose dehydrogenase, to produce the (S) amino acid.

15. The method of claim 14, wherein the amino acid is a compound of formula I:
the amino acid is a compound of formula (I) 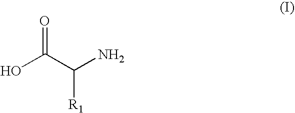
wherein R₁ is selected from the group consisting of alkenyl, alkyl, alkynyl, aryl, arylalkyl, cycloalkyl, cycloalkylalkyl, heteroaryl, and heteroarylalkyl.

16. The method of claim 15, wherein R₁ is heteroarylalkyl wherein the heteroaryl is pyridinyl optionally substituted with 2-methylphenyl.

17. The method of claim 15, wherein the amino acid is 2-RS-amino-3-[3-{6-(2-methylphenyl)}pyridyl]-propionic acid, or a salt thereof.

18. The method of claim 17 wherein the (S) enantiomer is 2-S-amino-3-[3-{6-(2-methylphenyl)}pyridyl]-propionic acid, or a salt thereof.

19. A method for converting a keto acid to an (S) amino acid, comprising reacting a keto acid of general formula 2: 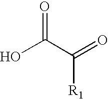
with (i) an amount of an aminotransferase of SEQ ID NO:2 in the presence of an amino acid, or (ii) an amino acid dehydrogenase of SEQ ID NO:4 in the presence of an amine donor to produce an (S) amino acid,
wherein R₁ is selected from the group consisting of alkenyl, alkyl, alkynyl, aryl, arylalkyl, cycloalkyl, cycloalkylalkyl, heteroaryl, and heteroarylalkyl. In a preferred embodiment, R₁ is heteroarylalkyl wherein the heteroaryl is pyridinyl optionally substituted with 2-methylphenyl.

20. The method of claim 19, wherein R₁ is heteroarylalkyl wherein the heteroaryl is pyridinyl optionally substituted with 2-methylphenyl.

21. A method for converting 2-RS-amino-3-[3-{6-(2-methylphenyl)}pyridyl]-propionic acid to S-Amino-[3-{6-(2-methylphenyl)}pyridyl]-propionic acid, comprising reacting 2-RS-amino-3-[3-{6-(2-methylphenyl)}pyridyl]-propionic acid with an amino acid oxidase in combination with a non-enzymatic reducing agent, or a salt thereof, in the presence of an inorganic catalyst, to produce S-amino-[3-{6-(2-methylphenyl)}pyridyl]-propionic acid.

22. The method of claim 21, wherein the non-enzymatic reducing agent is ammonia, or a salt thereof.

23. The method of claim 21, wherein the inorganic catalyst is a metal.

24. The method of claim 21 wherein the non-enzymatic reducing agent is selected from the group consisting of borohydride, borane ammonia, and a borane-amine complex, or salts thereof.
